(19) United States
(12) Patent Application Publication
(10) Pub. No.: US 2019/0071504 A1
(43) Pub. Date: Mar. 7, 2019

(54) Methods And Compositions For Treating And Preventing Disease Associated With Alpha 8 Beta 1 Integrin

(71) Applicant: The Regents of the University of California, Oakland, CA (US)

(72) Inventor: Kamran Atabai, Oakland, CA (US)

(21) Appl. No.: 16/084,944

(22) PCT Filed: Mar. 15, 2017
(86) PCT No.: PCT/US17/22446

§ 371 Date: Sep. 13, 2018

Related U.S. Application Data
(60) Provisional application No. 62/308,331, filed on Mar. 15, 2016.

(51) Int. Cl. C07K 16/28; A61K 45/06; A61K 39/395

(52) U.S. Cl. CPC C07K 16/2842; A61K 45/06; A61K 39/3955; C07K 2317/30; C07K 2317/24; C07K 2317/92; C07K 2317/76

(57) ABSTRACT

Provided herein are monoclonal antibodies that recognize, bind to, and block interactions of other molecules with integrin α8β1. Also provided herein are methods of using said antibodies to treat gastrointestinal motility disorders.

### CROSS-REFERENCE TO RELATED PATENT APPLICATIONS

The present patent application claims benefit of priority to U.S. Provisional Patent Application No. 62/308,331, filed Mar. 15, 2016, which is incorporated by reference.

### BACKGROUND OF THE INVENTION

Coordinated gastrointestinal smooth muscle contraction is critical for proper nutrient absorption. Smooth muscle function is altered in a number of medical disorders and secondary to commonly used medications leading to increased or decreased gastrointestinal motility. The RGD-binding integrin α8β1 is highly expressed in visceral smooth muscle where its function is unknown. The present invention demonstrates a critical role for α8β1 in promoting nutrient absorption through regulation of gastrointestinal motility. Smooth muscle specific deletion of α8 in the gastrointestinal tract in mice results in enhanced gastric antral smooth muscle contraction, more rapid gastric emptying of a food bolus, and more rapid transit of food through the small intestine leading to malabsorption of dietary fats and carbohydrates as well as protection from weight gain in a diet-induced model of obesity. Mechanistically, we identify the milk protein Mfge8 as a novel ligand for α8β1 and show that Mfge8 ligation of α8β1 reduces antral smooth muscle contractile force by preventing RhoA activation through a PTEN dependent mechanism. Collectively, our results identify a role for α8β1 in regulating gastrointestinal motility and identify α8 as a potential target for disorders characterized by hypo- or hypermotility. Hence, the integrin α8β1 may serve as a useful therapeutic target to treat gastrointestinal motility disorders.

### SUMMARY OF THE INVENTION

Herein, α8β1 was identified as the functional integrin receptor for Milk fat Globule Epidermal Growth Factor like 8 (Mfge8). Novel monoclonal blocking antibodies against α8β1 are provided herein as well as methods of their use in treating gastrointestinal disorders characterized by hypo- or hyper-motility.

In some embodiments, the present invention is directed towards an isolated or recombinant monoclonal antibody that specifically binds to a α8β1 polypeptide.

In some aspects, an antibody of the embodiments may be an IgG (e.g., IgG1, IgG2, IgG3 or IgG4), IgM, IgA, or an antigen binding fragment thereof. The antibody may be a Fab′, a F(ab′)2 a F(ab′)3, a monovalent scFv, a bivalent scFv, or a single domain antibody.

The antibody may be a human, humanized, or de-immunized antibody. In some aspects, the antibody may be conjugated to an imaging agent, a chemotherapeutic agent, a toxin, or a radionucleotide.

The invention provides an isolated antibody that binds with a high specificity or a high affinity to a protein having at least a 90% sequence identity to SEQ ID NO: 1. In a preferred embodiment, the isolated antibody binds with a high specificity or affinity to a protein having the sequence of SEQ ID NO: 1. The antibodies of the invention are used for the treatment of the gastrointestinal motility disorders in a subject described throughout this application. Those conditions include diabetic gastropathy, idiopathic gastroparesis, opioid-induced constipation, drug-induced ileus, idiopathic chronic constipation, intestinal pseudo-obstruction, bowel hypomotility, functional bowel disorders, constipation-predominant Irritable Bowel Syndrome, gastrointestinal-dysmotility, and obesity.

In some embodiments, invention provides a composition comprising an α8β1 binding antibody for use in the treatment of a gastrointestinal motility disorder in a patient or a subject. In other embodiments, the invention provides a composition for use in the manufacture of a drug for treating a gastrointestinal motility disorder in a patient or a subject. In a preferred embodiment, the antibody binds with a high affinity to a protein having at least a 90% sequence identity to SEQ ID NO: 1. In a more preferred embodiment, the antibody binds with a high affinity to a protein having the sequence of SEQ ID NO: 1.

The invention provides methods of treating patients, use in the treatment of patients, or use in the manufacture of a drug or medicament, with an antibody as described above and herein, that is a monoclonal antibody, a polyclonal antibody, a chimeric antibody, an affinity matured antibody, a humanized antibody, a human antibody, or an antigen-binding antibody fragment. In preferred embodiments, the antigen-binding fragment is a Fab, Fab′, Fab′-SH,F(ab′)z, or scFv.

In some embodiments, there is provided an isolated polynucleotide molecule comprising nucleic acid sequence encoding an antibody or a polypeptide comprising an antibody V_{H} or V_{L} domain disclosed herein.

In further embodiments, a host cell is provided that produces a monoclonal antibody or recombinant polypeptide of the embodiments. In some aspects, the host cell is a mammalian cell, a yeast cell, a bacterial cell, a ciliate cell, or an insect cell. In certain aspects the host cell is a hybridoma cell.

In still further embodiments, there is provided a method of manufacturing an antibody of the present invention comprising expressing one or more polynucleotide molecule(s) encoding a V_{L} or V_{H} chain of an antibody disclosed herein in a cell and purifying the antibody from the cell.

In additional embodiments, there are pharmaceutical compositions comprising an antibody or antibody fragment as discussed herein. Such a composition further comprises a pharmaceutically acceptable carrier and may or may not contain additional active ingredients.

In embodiments of the present invention, there is provided a method for treating a subject having a gastrointestinal disorder characterized by hypomotility comprising administering to the subject an effective amount of an agent that inhibits engagement of the α8β1 integrin receptor and its ligand, Mfge8. In one aspect, the agent may be an agent that disrupts the α8β1/Mfge8 interaction.

In embodiments of the present invention, there is provided a method for treating a subject having gastrointestinal disorders characterized by hypo-motility comprising administering an effective amount of an antibody disclosed herein.

In certain aspects, the gastrointestinal disorders are characterized by delayed motility leading to nausea, vomiting, and aspiration of stomach contents.

In one aspect, the antibody may be administered systemically. In additional aspects, the antibody may be administered intravenously, intradermally, intratumorally, intramuscularly, intraperitoneally, subcutaneously, anally, or orally. The method may further comprise administering at least a second gastrointestinal therapy to the subject. Examples of the second gastrointestinal therapy include, but are not limited to, surgical therapy, drug therapy, hormonal therapy, or cytokine therapy. In one aspect, the subject may be a human subject.

In further aspects, the method may further comprise administering a composition of the present invention more than one time to the subject, such as, for example, 1, 2, 3, 4, 5, 6, 7, 8, 9, 10, 15, 20 or more times.

In accordance with certain aspects of the present invention, there is provided a method for treating a gastrointestinal disorder comprising administering an effective amount of a α8β1-binding protein to treat a patient. In some aspects, a method comprises treating a patient who either has previously been determined to have a gastrointestinal disorder characterized by hypo- or hyper-motility, or is determined to have a gastrointestinal disorder characterized by hypo- or hyper-motility.

In accordance with certain aspects of the present invention, there is provided the use of a α8β1 binding antibody in the manufacture of a medicament for the treatment of a gastrointestinal motility disorder.

In certain embodiments, the α8β1-binding protein may be an antibody, which may be a monoclonal antibody, a polyclonal antibody, a chimeric antibody, an affinity matured antibody, a humanized antibody, a human antibody, or an antigen binding antibody fragment. Preferably, the antibody is a monoclonal antibody or a humanized antibody. In embodiments where the antibody is an antibody fragment, preferred fragments include Fab, Fab′, Fab′-SH, F(ab′)₂, or scFv molecules.

For certain medical or clinical applications, the antibody may be attached to an agent to be targeted to a α₈β₁-expressing cell. The agent may be a cytotoxic agent, a cytokine, an anti-angiogenic agent, a chemotherapeutic agent, a diagnostic agent, an imaging agent, a radioisotope, a pro-apoptosis agent, an enzyme, a hormone, a growth factor, a peptide, a protein, an antibiotic, an antibody, a Fab fragment of an antibody, an antigen, a survival factor, an anti-apoptotic agent, a hormone antagonist, a virus, a bacteriophage, a bacterium, a liposome, a microparticle, a nanoparticle, a magnetic bead, a microdevice, a cell, a nucleic acid, or an expression vector. Where the targeted molecule is a protein, the coding regions for the respective protein molecule and antibody may be aligned in frame to permit the production of a “fused” molecule where desired. In other embodiments, however, the antibody may be conjugated to the molecule using conventional conjugation techniques.

Certain embodiments are directed to an antibody or recombinant polypeptide composition comprising an isolated and/or recombinant antibody or polypeptide that specifically binds to the α8β1 integrin receptor. In certain aspects the antibody or polypeptide has a sequence that is, is at least, or is at most 80, 85, 90, 95, 96, 97, 98, 99, or 100% identical (or any range derivable therein) to all or part of any monoclonal antibody provided herein.

In yet further aspects, an antibody or polypeptide of the embodiments comprises an amino acid segment that is at least 80, 85, 90, 95, 96, 97, 98, 99, or 100% identical (or any range derivable therein) to a V, VJ, VDJ, D, DJ, J or CDR domain of an anti-α8β1 antibody. For example, a polypeptide may comprise 1, 2 or 3 amino acid segments that are at least 80, 85, 90, 95, 96, 97, 98, 99, or 100% identical (or any range derivable therein) to CDRs 1, 2, and/or 3 of an anti-α8β1 antibody.

In one embodiment, a composition comprising an anti-α8β1 antibody is provided for use in the treatment of a gastrointestinal disorder in a patient. In another embodiment, the use of an anti-α8β1 antibody in the manufacture of a medicament for the treatment of a gastrointestinal disorder is provided.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic acid are used to make the invention readily understandable by the skilled artisan; however, these terms may be used interchangeably with “comprise” or “comprising,” respectively.

As used herein the specification. “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1G. Mfge8 regulates gastrointestinal motility. (FIG. 1A) Force of antral smooth muscle ring contraction with and without the addition of rMfge8 or RGE construct in Mfge8^{-/-} and Mfge8^{+/+} in response to MCh (N=4-5). (FIG. 1B) Force of antral smooth muscle ring contraction with and without the addition of rMfge8 or RGE construct in Mfge8^{-/-} and Mfge8^{+/+} in response to KCl (N=4-5). (FIG. 1C) Force of antral smooth muscle ring contraction after in vivo induction of smooth muscle Mfge8 expression in Mfge8^{-/-}sm+ mice in response to MCh (N=5). (FIG. 1D) The rate of gastric emptying in Mfge8^{-/-} and Mfge8^{-/-} with and without the addition of rMfge8 or RGE construct (N=10). (FIG. 1E) The rate of gastric emptying after smooth muscle transgenic (Mfge8−/−sm+) expression of Mfge8 (N=7). (FIG. 1F) Small intestinal transit time in Mfge8^{-/-} and Mfge8^{+/+} with and without the addition of rMfge8 or RGE construct (N=5-10). (FIG. 1G) Small intestinal transit time after smooth muscle transgenic expression of Mfge8 (N=4-5). Female mice were used for all experiments in FIG. 1. *P<0.05, **P<0.01, ***P<0.001. Data are expressed as mean±s.e.m.

FIGS. 2A-2K. Mfge8 binds to α8 integrin to regulate gastrointestinal motility. (FIG. 2A) Purified α8, αvβ3, or α5β1 were used for solid-phase binding assays with purified Mfge8 at indicated concentrations in the presence or absence of 10 mM EDTA. (FIG. 2B) Adhesion of SW480 (mock), α8 transfected SW480 cells (α8) or β3 transfected SW480 cells (β3) adhesion to wells coated with rMfge8 (5 μg/ml) in the presence or absence of integrin blocking antibodies (5 μg/ml) against β5 (ALULA), β3 (LM609) or α8 (YZ83). (FIG. 2C) Dose-dependent binding of SW480 cells to wells coated with a dose range of rMfge8 in the presence of a 35 blocking antibody. (FIG. 2D) Western blot of integrin expression in human gastric smooth muscle cells (HGSMC), SW480 cells and α8 transfected SW480 (SW480_α8) cells. (FIG. 2E) Human gastric smooth muscle cell adhesion to rMfge8-coated wells in the presence of blocking antibodies against the αv, β1, β5, α8, or α5 integrin subunits. (FIG. 2F) Force of antral contraction in WT and α8sm^{-/-} mice in response to MCh (N=3-4). (FIG. 2G) The rate of gastric emptying in α8sm^{-/-} and WT mice with and without the addition of rMfge8 (N=4-5). (FIG. 2H) Small intestinal transit time in α8sm^{-/-} and WT mice with and without the addition of rMfge8 (N=4-5). (FIG. 2I) Force of antral contraction in WT mice after IP injection of α8 blocking or control antibody in response to MCh (N=4-5). (FIG. 2J) The rate of gastric emptying in WT mice after IP injection of α8 blocking or IgG1 isotype control antibody (N=7). (FIG. 2K) Small intestinal transit time in WT mice after IP injection of α8 blocking or IgG1 isotype control antibody (N=7). *P<0.05, **P<0.01, ***P<0.001. Data are expressed as mean±s.e.m.

FIGS. 3A-3C. α8 integrin regulates antrum smooth muscle calcium sensitivity by preventing RhoA activation. (FIG. 3A) Force of antral smooth muscle ring contraction with and without the addition of ROCK inhibitor Y-27632 (N=3-4). (FIG. 3B) The rate of gastric emptying in Mfge8^{-/-} and Mfge8^{+/+} with and without the IP injection of ROCK inhibitor (Y-27632) or control inhibitor (N=5-11). (FIG. 3C) Small intestinal transit times Mfge8^{-/-} and Mfge8^{+/+} with and without IP injection of ROCK inhibitor (Y-27632) or control inhibitor (N=6-11). Female mice were used for all experiments. *P<0.05, **P<0.01, ***P<0.001. Data are expressed as mean±s.e.m.

FIGS. 4A-4B. Mfge8 ligation of α8β1 integrin inhibits PI3 kinase activity. (FIG. 4A) Force of antral smooth muscle ring contraction with and without the addition of PI3K inhibitor wortmannin (wort 100 ng/ml) in response to MCh in WT and Mfge8−/− (N=4-5) (FIG. 4B) Force of antral smooth muscle ring contraction with and without the addition of PI3K inhibitor wortmannin (wort 100 ng/ml) in response to MCh in WT and α8sm−/− (N=4-5). *P<0.05, **P<0.01, ***P<0.001. Data are expressed as mean±s.e.m.

FIGS. 5A-SD. Mfge8 modulates PTEN activity. (FIG. 5A) PTEN activity in antral smooth muscle of WT and Mfge8−/− (N=5) with and without the addition of rMfge8 and RGE construct. (FIG. 5B) PTEN activity in antral smooth muscle of WT and α8sm−/− (N=7) with and without the addition of rMfge8 and RGE construct. (FIG. 5C) PTEN activity in antral smooth muscle strips of WT mice after IP injection of α8 blocking or IgG1 isotype control antibody. (N=5). (SD) Western blot of human gastric smooth muscle cells (HGSMC) treated with PTEN siRNA and with 5-HT demonstrating active and total RhoA using a GST pull-down assay. *P<0.05, **P<0.01, ***P<0.001. Data are expressed as mean±s.e.m.

FIG. 6A-6J. α8sm−/− mice are protected from diet-induced obesity. (FIG. 6A) Fecal triglycerides in WT and α8sm−/− mice after an olive oil gavage (N=8). (FIG. 6B) Serum triglycerides levels in WT and α8sm−/− mice after an olive oil gavage (N=5). (FIG. 6C) Fecal triglycerides in WT and α8sm−/− mice on a normal chow control diet (N=6). (FIG. 6D) Fecal (N=8) 2NBDG content in WT and α8sm−/− mice after gavage with a 2NBDG-methylcellulose mixture. (FIG. 6E) Enterocyte (N=8) 2NBDG content in WT and α8sm−/− mice after gavage with a 2NBDG-methylcellulose mixture. (FIG. 6F) Fecal (N=8) 2NBDG content in WT and Mfge8−/− mice after gavage with a 2NBDG-methylcellulose mixture. (FIG. 6G) Enterocyte (N=8) 2NBDG content in WT and Mfge8−/− mice after gavage with a 2NBDG-methylcellulose mixture. (FIG. 6H) Weight gain in female WT and α8sm−/− mice on a normal chow diet (CD) (N=6-8) or HFD (N=8-12). (FIG. 6I) Fecal energy content in WT and α8sm−/− mice on a normal chow diet (CD) (N=5-6) or HFD (N=4-5). Each sample represents stool combined from 3 mice. Female mice were used for all experiments. (FIG. 6J) Fecal triglycerides in WT and β3/β5 integrin-deficient mice with normal chow control diet (N=5-6). For all in vivo experiments, each group of 5 mice represents 1 independent experiment. *P<0.05, **P<0.01, ***P<0.001. Data are expressed as mean±s.e.m.

FIGS. 7A-7C 2. Normal gastrointestinal motility in β3−/−, β5−/− and β3/β5−/− mice. (FIG. 7A) Force of antral smooth muscle ring contraction in β3−/−, β5−/− and β3/β5−/− mice in response to MCh. (FIG. 7B) The rate of gastric emptying in β3−/−, β5−/− and β3/β5−/− mice with and without the addition of rMfge8 (N=5-6). (FIG. 7C) Small intestinal transit time in β3−/−, β5−/− and β3/β5−/− mice with and without the addition of rMfge8 (N=5-6). P<0.05, **P<0.01, ***P<0.001. Data are expressed as mean±s.e.m.

FIG. 8. Mfge8 increases PTEN activity but not other binding partners of α8 integrin. PTEN activity assay in Human Gastric Smooth Muscle Cells after treatment with rMfge8. RGE construct, fibronectin or vitronectin (N=5). **P<0.01, ***P<0.001. Data are expressed as mean±s.e.m.

FIGS. 9A-9C. Protection from weight gain in α8sm−/− mice on a HFD. (FIG. 9A) Weight gain in 1WT and α8sm−/− male mice on a CD (N=6-8) or HFD (N=8-10). Body composition of WT and α8sm−/− mice aged 14 weeks on a HFD (FIG. 9B, N=8-12) or on a CD (FIG. 9C, N=6-8). *P<0.05, **P<0.01. Data are expressed as mean t s.e.m.

### DESCRIPTION OF ILLUSTRATIVE EMBODIMENTS

The present invention is based, in part, on the finding that RGD-binding integrin α8β1 is highly expressed in visceral smooth muscle and plays a critical role in promoting nutrient absorption through regulation of gastrointestinal motility. The integrin receptor α8β1 is the cell surface receptor for the milk protein, Mfge8. Monoclonal antibodies against α8β1 results in enhanced gastric antral smooth muscle contraction, more rapid gastric emptying of a food bolus, and more rapid transit of food through the small intestine leading to malabsorption of dietary fats and carbohydrates as well as protection from weight gain. These results suggest that the α8β1/Mfge8 interaction is a target with therapeutic potential for disorders characterized by hypo- or hypermotility.

### I. Mfge8 and α8β1

Milk fat Globule Epidermal Growth Factor like 8 (Mfge8) is an integrin ligand that is highly expressed in breast milk. Mfge8 coordinates absorption of dietary fats by promoting enterocyte fatty acid uptake after ligation of the αvβ3 and αvβ5 integrins. Mfge8 also modulates smooth muscle contractile force. In mice deficient in Mfge8 (Mfge8^{-/-}), airway and jejunal smooth muscle contraction is enhanced in response to contractile agonists after these muscle beds have been exposed to inflammatory cytokines but not under basal conditions. Contraction of antral smooth muscle is a key determinant of the rate at which a solid food bolus exits the stomach and transits through the primary site of nutrient absorption, the small intestine. Since Mfge8 promotes enterocyte fatty acid uptake and can regulate smooth muscle contraction, we were interested in examining whether Mfge8 reduces the force of basal antral smooth muscle contraction, thereby slowing gastrointestinal motility and allowing a greater time for nutrient absorption.

α8β1 is a member of the RGD binding integrin family and is prominently expressed in smooth muscle. The most definitive in vivo role described for α8β1 is in kidney morphogenesis where deletion of this integrin subunit leads to impaired recruitment of mesenchymal cells into epithelial structures. Osteopontin, fibronectin, vitronectin, nephronectin, and tenascin-C have all previously been identified as ligands for α8β1. In this work we show that Mfge8 is a novel ligand for α8β1 and that Mfge8 ligation of α8β1 reduces the force of gastric antral smooth muscle contraction and the rate of gastric emptying and increases small intestinal transit time. We further show that mice with smooth muscle specific deletion of α8 integrin subunit (α8sm^{-/-}) develop malabsorption of ingested fats and carbohydrates and are partially protected from weight gain in a model of diet-induced obesity. α8β1 slows gastrointestinal motility by increasing the activity of Phosphatase and tensin homolog (PTEN) leading to reduced activation of the Ras homolog gene family member RhoA.

### II. Therapeutic Antibodies

In certain embodiments, an antibody or a fragment thereof that binds to at least a portion of α8β1 protein and inhibits Mfge8/α8β1 binding and its associated use in treatment of diseases are contemplated. As used herein, the term “antibody” is intended to refer broadly to any immunologic binding agent, such as IgG, IgM, IgA. IgD, and IgE as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Preferably, the anti-α8β1 antibody is a monoclonal antibody or a humanized antibody. By known means and as described herein, polyclonal or monoclonal antibodies, antibody fragments, and binding domains and CDRs (including engineered forms of any of the foregoing) may be created that are specific to α8β1 protein, one or more of its respective epitopes, or conjugates of any of the foregoing, whether such antigens or epitopes are isolated from natural sources or are synthetic derivatives or variants of the natural compounds.

The term antibody is meant to include monoclonal antibodies, polyclonal antibodies, toxin-conjugated antibodies, drug-conjugated antibodies (ADCs), humanized antibodies, antibody fragments (e.g., Fc domains), Fab fragments, single chain antibodies, bi- or multi-specific antibodies, Llama antibodies, nano-bodies, diabodies, affibodies, Fv, Fab, F(ab′)2, Fab′, scFv, scFv-Fc, and the like. Also included in the term are antibody-fusion proteins, such as Ig chimeras. Preferred antibodies include humanized or fully human monoclonal antibodies or fragments thereof.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region. “Antibody fragments” comprise a portion of an intact antibody, preferably comprising the antigen binding region thereof. Examples of antibody fragments include Fab, Fab′. F(ab′)2, and Fv fragments; diabodies; linear antibodies; single-chain antibody molecules; and multispecific antibodies formed from antibody fragments. The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies.

In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations that typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

Antibodies that bind specifically to an antigen have a high affinity for that antigen. Antibody affinities may be measured by a dissociation constant (Kd). In certain embodiments, an antibody provided herein has a dissociation constant (Kd) of equal to or less than about 100 nM, 10 nM, 1 nM, 0.1 nM, 0.01 nM, or 0.001 nM (e.g. 10⁻⁷ M or less, from 10⁻⁷ M to 10⁻¹³ M, from 10⁻⁸ M to 10⁻¹³ M or from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20®; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with, e.g., immobilized antigen CM5 chips at ^{˜}10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (^{˜}0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (Kₒₙ) and dissociation rates (K_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See. e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette. Other coupling chemistries for the target antigen to the chip surface (e.g., streptavidin/biotin, hydrophobic interaction, or disulfide chemistry) are also readily available instead of the amine coupling methodology (CM5 chip) described above, as will be understood by one of ordinary skill in the art.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al, Nature, 256: 495 (1975); Harlow et al, Antibodies: A Laboratory Manual, (Cold Spring Harbor Laboratory Press, 2nd ed. 1988): Hammerling et al., in: Monoclonal Antibodies and T-Cell Hybridomas pp. 563-681 (Elsevier, N.Y., 1981)), recombinant DNA methods (see, e.g., U.S. Pat. No. 4,816,567), phage display technologies (see. e.g., Clackson et al., Nature, 352: 624-628 (1991): Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., W098/24893; WO96/34096; W096/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger. Nature Biotechnol. 14: 826 (1996) and Lonberg and Huszar, Intern. Rev. Immunol. 13: 65-93 (1995). The above patents, publications, and references are incorporated by reference in their entirety.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23: 1035-1038 (1995); Hurle and Gross, Curr. Op. Biotech. 5:428-433 (1994). The foregoing references are incorporated by reference in their entirety.

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see. e.g., Marks et al., J. Mol. Biol, 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor, J. Immunol, 133: 3001 (1984); Brodeur et al., Monoclonal Antibody Production Techniques and Applications, pp. 55-93 (Marcel Dekker, Inc., New York, 1987); and Boerner et al., J. Immunol, 147: 86 (1991)); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci USA, 90: 2551 (1993): Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

Examples of antibody fragments suitable for the present embodiments include, without limitation: (i) the Fab fragment, consisting of V_{L}, V_{H}. C_{L}, and CH₁ domains; (ii) the “Fc” fragment consisting of the V_{H} and C_{H1} domains; (iii) the “Fv” fragment consisting of the VL and VH domains of a single antibody; (iv) the “dAb” fragment, which consists of a V_{H} domain; (v)isolated CDR regions: (vi) F(ab′)2 fragments, a bivalent fragment comprising two linked Fab fragments; (vii) single chain Fv molecules (“scFv”), wherein a VH domain and a VL domain are linked by a peptide linker that allows the two domains to associate to form a binding domain; (viii) bi-specific single chain Fv dimers (see U.S. Pat. No. 5,091,513); and (ix) diabodies, multivalent or multispecific fragments constructed by gene fusion (US Patent App. Pub. 20050214860). Fv, scFv, or diabody molecules may be stabilized by the incorporation of disulfide bridges linking the VH and VL domains. Peptibodies comprising a scFv joined to a C_{H3} domain may also be made.

Antibody-like binding peptidomimetics are also contemplated in embodiments that describe “antibody like binding peptidomimetics” (ABiPs). These are peptides that act as pared-down antibodies and have certain advantages of longer serum half-life as well as less cumbersome synthesis methods.

Integrin α8 human protein sequence (SEQ ID NO: 1) and integrin α8 mouse protein sequence (SEQ ID NO: 2) may be used to produce human recombinant proteins and peptides as is well known to people skilled in the art. Integrin α8 human mRNA sequence (SEQ ID NO: 3) and integrin α8 mouse mRNA sequence (SEQ ID NO: 4) may be used to produce mouse recombinant proteins and peptides as is well known to people skilled in the art. Integrin β1 human protein sequence (SEQ ID NO: 5) may be used to produce human recombinant proteins and peptides as is well known to people skilled in the art. For example, such mRNA sequences could be engineered into a suitable expression system, e.g., yeast, insect cells, or mammalian cells, for production of a α8 protein or peptide.

Animals may be inoculated with an antigen, such as a soluble α8β1 protein, in order to produce antibodies specific for α8β1 protein. Frequently an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

A monoclonal antibody is a single species of antibody wherein every antibody molecule recognizes the same epitope because all antibody producing cells are derived from a single B-lymphocyte cell line. The methods for generating monoclonal antibodies (mAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice (e.g., BALB/c mice) are routinely used and generally give a high percentage of stable fusions. Hybridoma technology involves the fusion of a single B lymphocyte from a mouse previously immunized with a α8β1 antigen with an immortal myeloma cell (usually mouse myeloma). This technology provides a method to propagate a single antibody producing cell for an indefinite number of generations, such that unlimited quantities of structurally identical antibodies having the same antigen or epitope specificity (monoclonal antibodies) may be produced.

In one embodiment, the antibody is a chimeric antibody, for example, an antibody comprising antigen binding sequences from a non-human donor grafted to a heterologous nonhuman, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; 6,891,024; 7,407,659; and 8,178,098. All patents, patent application publications, and other publications cited herein are incorporated by reference in their entirety.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference.

It is fully expected that antibodies to α8β1 will have the ability to block α8β1 binding regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragments, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described above, antibodies may also be constructed so as to be chimeric or partially or fully human, so as to reduce or eliminate the adverse immunological consequences resulting from administering to an animal an antibody that has been produced in, or has sequences from, other species.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinantory recombinant protein may be isolated from bacteria. It is also contemplated that a bacterium containing such a variant may be implemented m compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds α8β1.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against a α8β1 polypeptide and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photo affinity molecules, colored particles or ligands, such as biotin.

Several methods are known in the art for the attachment or conjugation of an antibody to its conjugate moiety. Some attachment methods involve the use of a metal chelatecomplex employing, for example, an organic chelating agent such adiethylenetriamine pentaacetic acid anhydride (DTPA); ethylenetriamine tetraacetic acid; Nchloro-p-toluene sulfonamide; and/or tetrachloro-3-6-diphenylglycouril attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

### III. Treatment of Diseases

Certain aspects of the present embodiments can be used to prevent or treat a gastrointestinal disease or disorder associated with an Mfge8/α8β1 interaction. Functioning of the Mfge8/α8β1 ligation may be reduced by any suitable drugs to prevent the Mfge8/α8β1 ligation. These substances can be natural products or synthetic, they can be small chemical compounds, large molecules such as peptides, peptidomimetics or antibodies, small interfering RNAs (siRNAs), and anti-sense RNAs. Preferably, such substances would be an anti-α8β1 antibody.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that inhibits the Mfge8/α8β1 ligation.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a gastrointestinal disease.

An antibody that binds to α8β1 may be administered to treat a gastrointestinal disorder. Additional individuals to which blocking α8β1 antibodies can be administered include individuals having diabetic gastropathy (including gastroparesis), idiopathic gastroparesis, opioid-induced constipation, drug-induced ileus (for example, narcotics), idiopathic chronic constipation, intestinal pseudo-obstruction, bowel hypomotility, functional bowel disorders, and gastrointestinal-dysmotility secondary to systemic sclerosis (scleroderma).

A. Pharmaceutical Preparations

Where clinical application of a therapeutic composition containing an inhibitory antibody is undertaken, it will generally be beneficial to prepare a pharmaceutical or therapeutic composition appropriate for the intended application. This will typically entail preparing a pharmaceutical composition that is essentially free of pyrogens, as well as any other impurities that could be harmful to humans or animals. One may also employ appropriate buffers to render the complex stable and allow for uptake by target cells. In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired.

The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/bodyweight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions, solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions, formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaccous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic base such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Solutions of therapeutic compositions can be prepared in water suitably mixed with a surfactant, such as hydroxypropyl cellulose. Dispersions also can be prepared in glycerol, liquid polyethylene glycols, mixtures thereof, and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters, such as ethyloleate. Aqueous carriers include water, alcoholic/aqueous solutions, saline solutions, parenteral vehicles, such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

The therapeutic compositions of the present invention may include classic pharmaceutical preparations. Administration of therapeutic compositions according to the present invention will be via any common route so long as the target tissue is available via that route. This includes oral, nasal, buccal, rectal, vaginal or topical. Alternatively, administration may be orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, or respiratory tract, aerosol delivery can be used. Volume of the aerosol is between about 0.01 mL and 0.5 mL.

An effective amount of the therapeutic composition is determined based on the intended goal. For example, one skilled in the art can readily determine an effective amount of an antibody of the invention to be administered to a given subject, by taking into account factors such as the size and weight of the subject; the extent of the neovascularization or disease penetration; the age, health and sex of the subject; the route of administration; and whether the administration is regional or systemic. The term “unit dose” or “dosage” refers to physically discrete units suitable for use in a subject, each unit containing a predetermined-quantity of the therapeutic composition calculated to produce the desired responses discussed above in association with its administration, i.e., the appropriate route and treatment regimen. The quantity to be administered, both according to number of treatments and unit dose, depends on the protection or effect desired.

Precise amounts of the therapeutic composition also depend on the judgment of the practitioner and are particular to each individual. Factors affecting the dose include the physical and clinical state of the patient, the route of administration, the intended goal of treatment (e.g., alleviation of symptoms versus cure) and the potency, stability and toxicity of the particular therapeutic substance.

B. Combination Treatments

In certain embodiments, the compositions and methods of the present embodiments involve an antibody or an antibody fragment against α8β1 to inhibit the α8β1/Mfge8 interaction, in combination with a second or additional therapy. Such therapy can be applied in the treatment of any gastrointestinal disease that is associated with a α8β1/Mfge8 interaction.

For the treatment of idiopathic and diabetic gastroparesis, an antibody or an antibody fragment against α8β1 can be used alone or in combination with prokinetic agents (metoclopramide, erythromycin, domperidone, and other D2 dopaminergic antagonists, and ghrelin agonists) as a second or additional therapy.

For functional gastrointestinal disorders, which include chronic idiopathic constipation, constipation predominant irritable bowel syndrome (IBS-C), an antibody or an antibody fragment against α8β1 can be used either alone or in combination with bulk agents, for example, bran, laxatives, cathartics, for example, magnesium salts, stool softeners and lubricants, for example, docusates, and Prokinetic agents, disclosed herein, in addition to cholinomimetics, opioid antagonists, misoprostol, neurotrophin NT3, and new 5HT4 agonists such as prucalopride.

The methods and compositions disclosed herein, including combination therapies, enhance the therapeutic or protective effect, and/or increase the therapeutic effect of another gastrointestinal therapy.

### IV. Kits and Diagnostics

In various aspects of the embodiments, a kit is envisioned containing therapeutic agents and/or other therapeutic and delivery agents. In some embodiments, a kit is contemplated for preparing and/or administering a therapy of the embodiments. The kit may comprise one or more sealed vials containing any of the pharmaceutical compositions of the present embodiments. The kit may include, for example, at least one anti-α8β1 antibody as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

In order that the invention described herein may be more fully understood, the following examples are set forth. It should be understood that these examples are for illustrative purposes only and are not to be construed as limiting this invention in any manner.

### EXAMPLES

### Example 1—Mfge8 Regulates Gastrointestinal Motility

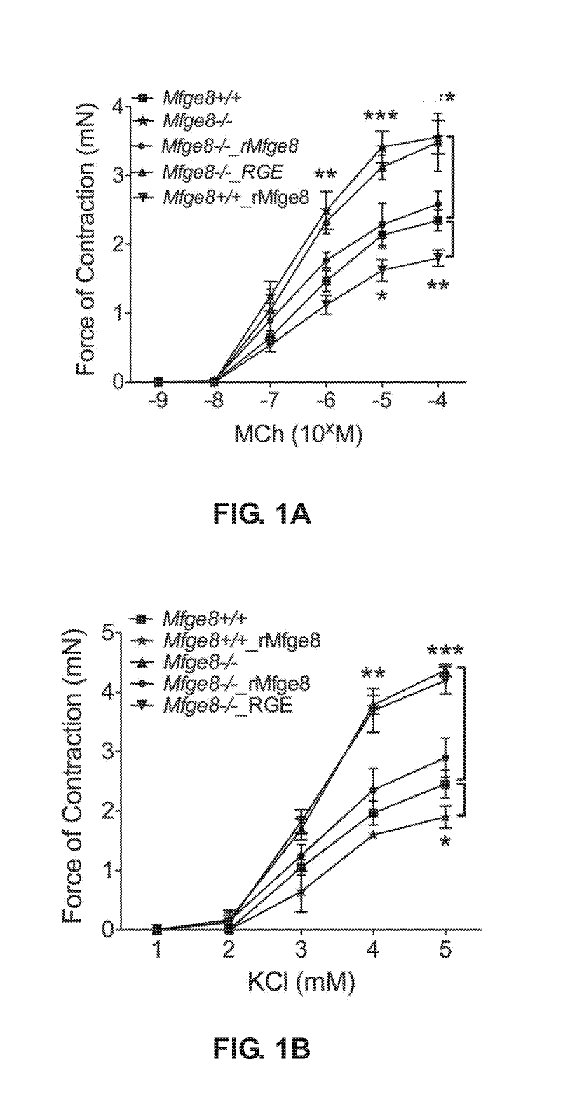

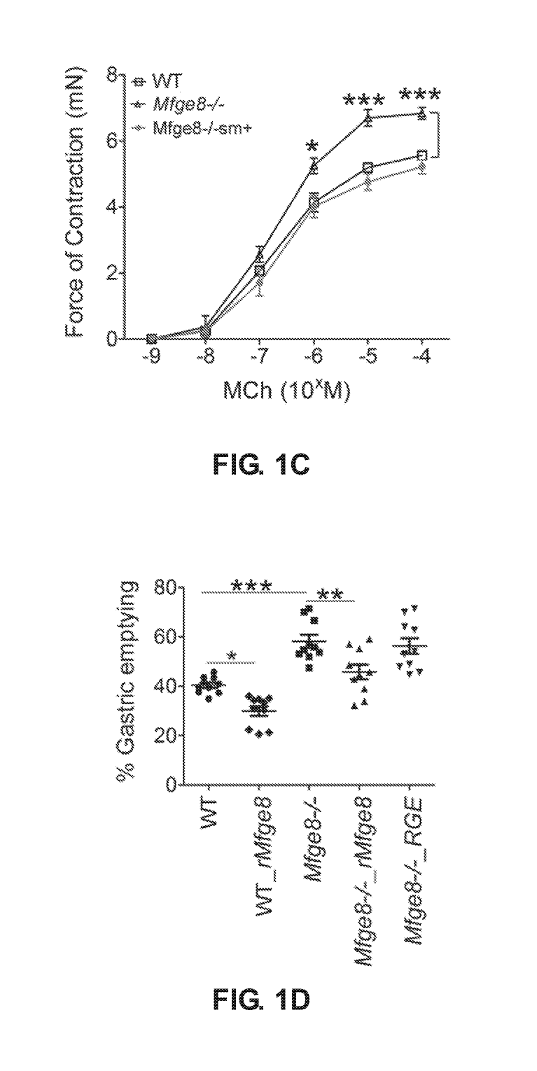

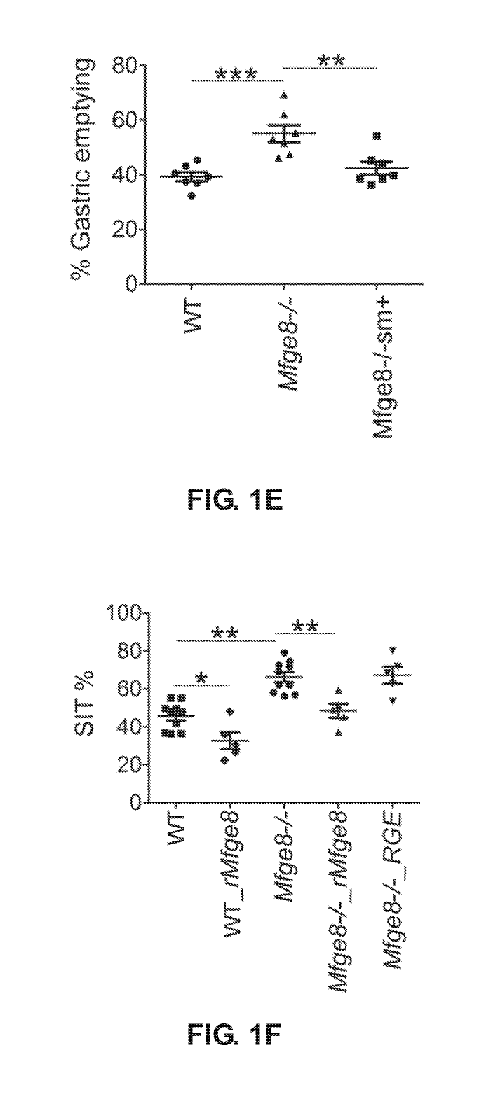

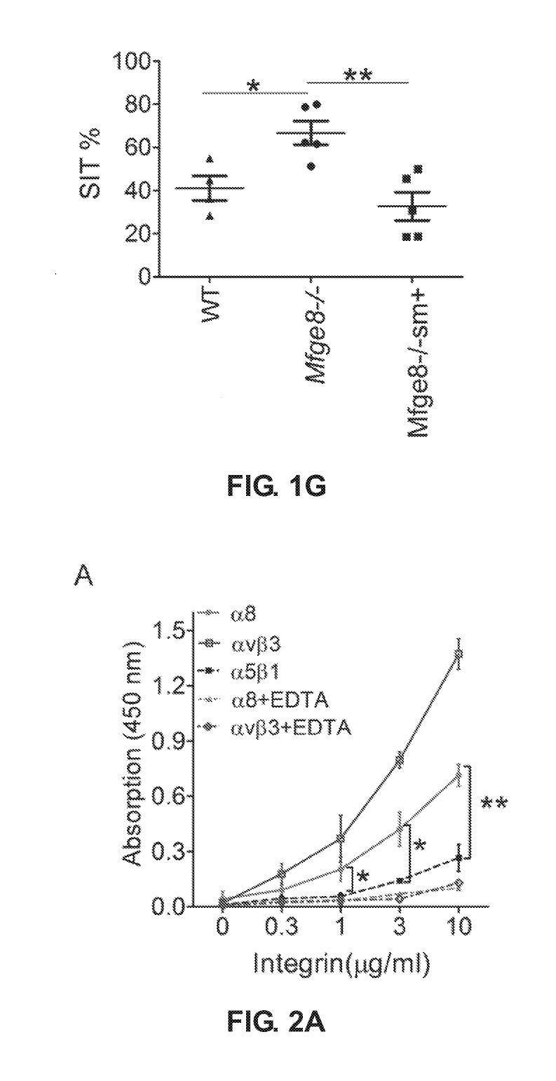

To determine whether Mfge8 regulates the force of antral smooth muscle contraction, we isolated gastric antral rings and measured the force of antral contraction in a muscle bath. Antral rings isolated from Mfge8^{-/-} mice had increased force of contraction in response to both methacholine (MCh) and KCl as compared with wild type (WT) controls (FIG. 1A,B). Incubation with recombinant Mfge8 (rMfge8), but not a recombinant construct where the integrin binding RGD sequence was mutated to RGE, rescued enhanced contraction indicating that the effect of Mfge8 on gastric smooth muscle was integrin-dependent (FIG. 1A, B). Induction of Mfge8 expression in the smooth muscle of a Mfge8^{-/-} transgenic mouse line where Mfge8 expression was driven by a tetracycline-inducible Mfge8 construct coupled with an α-smooth muscle-rtTA construct (Mfge8^{-/-}sm⁺) also rescued enhanced contraction (FIG. 1C). We next determined whether enhanced antrum contractility was associated with altered gastric emptying and small intestinal transit times (SIT), two functional in vivo measures of gastrointestinal motility. Mfge8^{-/-} mice had significantly more rapid gastric emptying and SIT (FIG. 1D-G). Administration of rMfge8 by gavage and transgenic smooth muscle expression of Mfge8 significantly reduced the rate of gastric emptying and SIT in Mfge8^{-/-} mice (FIG. 1D-G). Administration of rMfge8 by gavage also significantly reduced gastric emptying and SIT in WT mice (FIGS. 1D and 1F).

Enhanced antral smooth muscle contraction could be the result of an increase in the frequency of intracellular calcium oscillations after release of calcium from intracellular sources or from an increase in calcium sensitivity due to inactivation of the enzyme myosin light chain phosphatase²³⁻²⁵. Antral rings from Mfge8^{-/-} mice had exaggerated contraction to both MCh and KCl suggesting altered calcium sensitivity as the mechanism by which Mfge8 reduced contraction since these agonists increase intracellular calcium through different mechanisms. KCl works primarily by inducing opening of voltage gated calcium channels leading to influx of extracellular calcium while MCh induces release of intracellular calcium stores after receptor binding. To determine whether enhanced antral contraction was due to an increase in smooth muscle calcium sensitivity, we assessed the phosphorylation status of the regulatory subunit of myosin light chain phosphatase, MYPT, and myosin light chain (MLC)^{23,26}. Antral smooth muscle from Mfge8^{-/-} mice had increased phosphorylation of both MYPT and MLC in response to MCh as compared with WT smooth muscle.

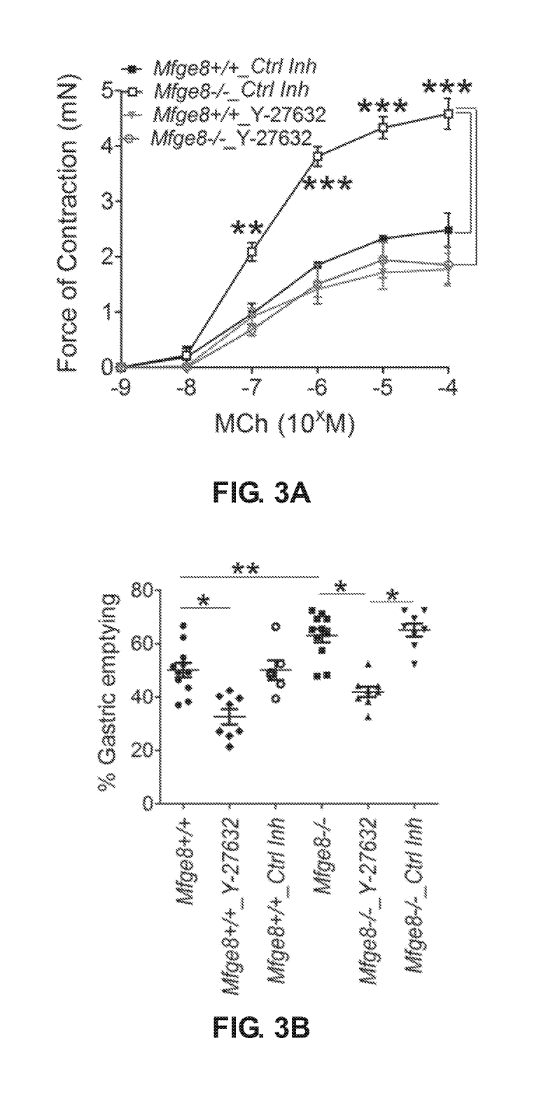

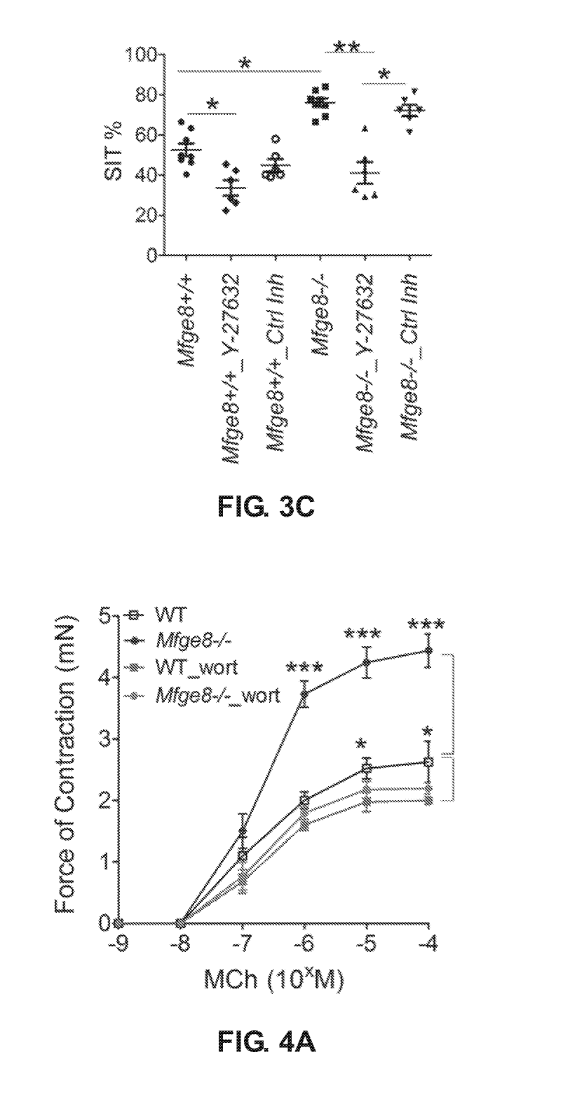

The small GTPase RhoA is a prominent regulator of MYPT phosphorylation and inhibition of RhoA has been shown to reduce the force of gastric smooth muscle contraction²⁷⁻²⁹. RhoA activation, assessed by a GST pull-down assay, was significantly increased in Mfge8^{-/-} antral smooth muscle as compared with WT controls while total RhoA protein expression was unchanged. rMfge8 reduced RhoA activation in WT and Mfge8^{-/-} antral smooth muscle. Pharmacological inhibition of ROCK, the kinase downstream of RhoA responsible for phosphorylation and inactivation of MYPT, with Y-27632, inhibited antral contraction in both WT and Mfge8^{-/-} smooth muscle reducing Mfge8^{-/-} antral contraction to WT levels (FIG. 3A). IP Y-27632 also reduced gastric emptying and SIT in WT and Mfge8^{-/-} mice with a relatively greater effect in Mfge8^{-/-} mice (FIGS. 3B and 3C). Taken together, these data indicate that in gastric antral smooth muscle, Mfge8 prevents RhoA activation leading to reduced smooth muscle calcium sensitivity, antral contraction, gastric emptying, and small intestinal transit times.

### Example 2—Mfge8 is a Ligand for the α8β1 Integrin

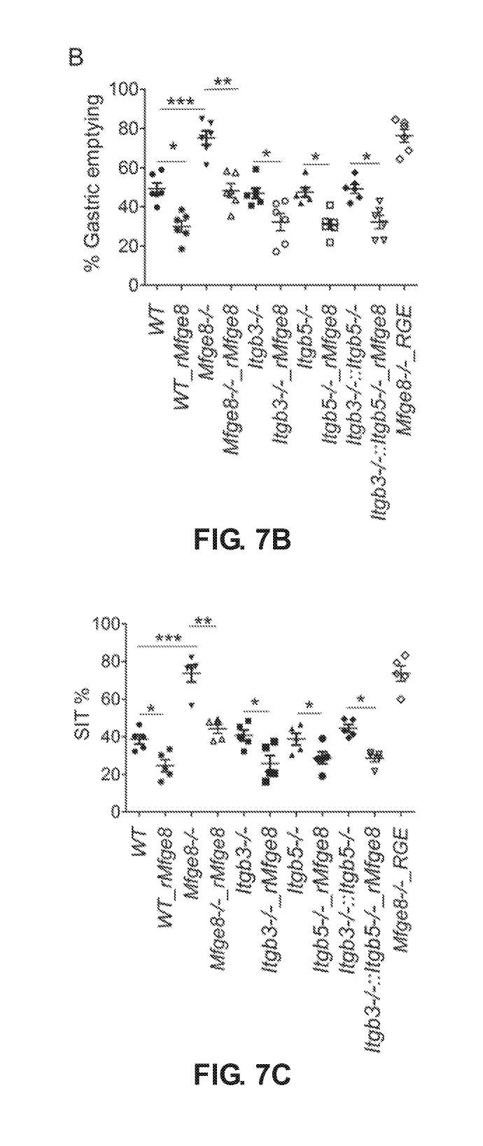

The αvβ3 and αvβ5 integrins are the known cell surface receptors for Mfge8^{9,30,31} and mediate the effect of Mfge8 on fatty acid uptake¹⁰. We therefore investigated whether these integrins mediated the effect of Mfge8 on gastrointestinal motility. Antrum contraction was similar in WT, β3^{-/-}, β5^{-/-} and β3β5^{-/-} mice (FIG. 7A). Gastric emptying and SIT was also similar in β3^{-/-}, β5^{-/-} and β3β5^{-/-} mice and rMfge8 significantly reduced the rate of gastric emptying and SIT in each mouse line (FIGS. 7B and 7C). rMfge8 also reduced MYPT and MLC phosphorylation in response to MCh to a similar extent in antrum smooth muscle from WT and β3β5^{-/-} mice. These data indicate that the effect of Mfge8 on smooth muscle contraction occurs via a novel RGD-binding integrin partner.

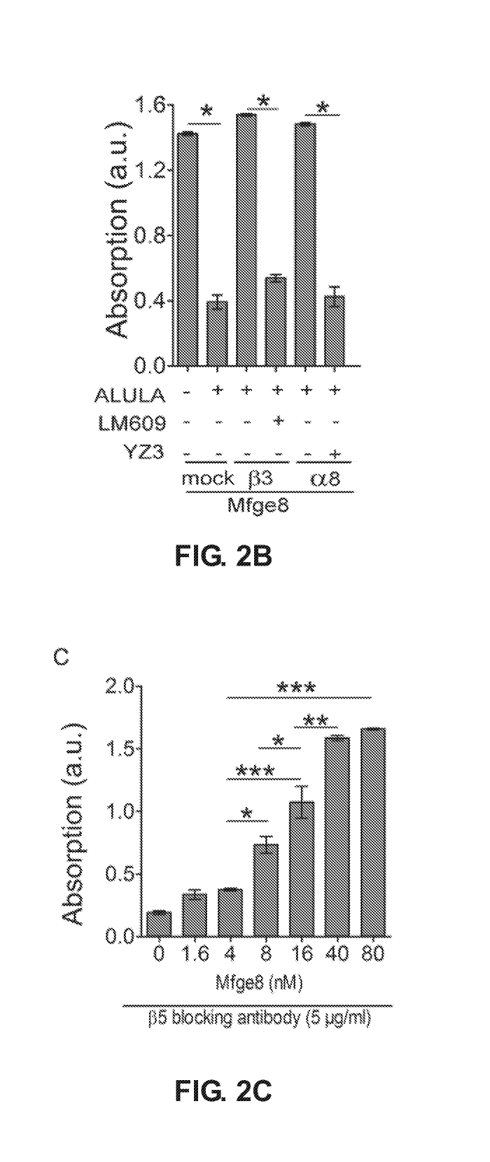

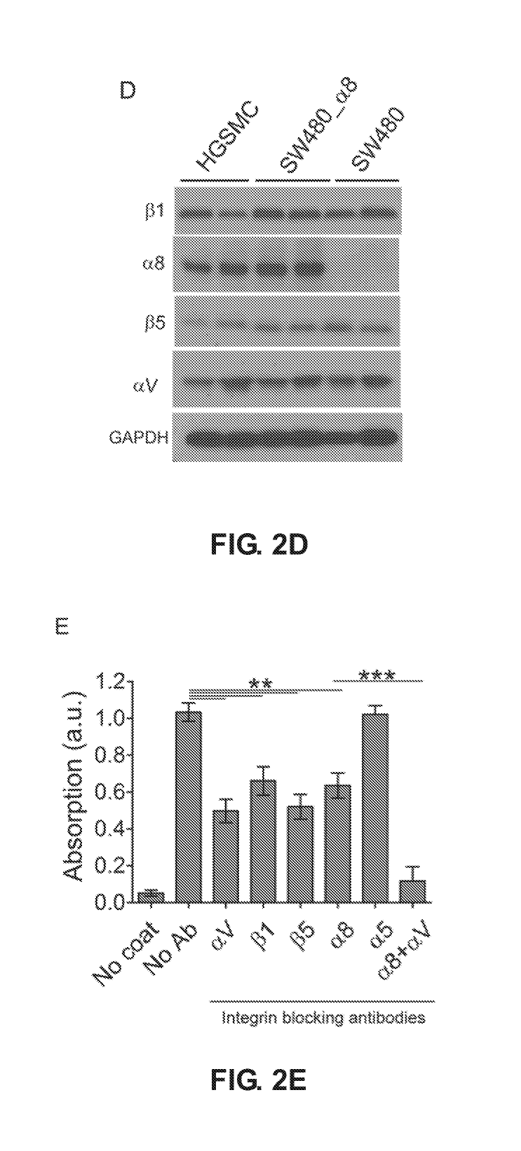

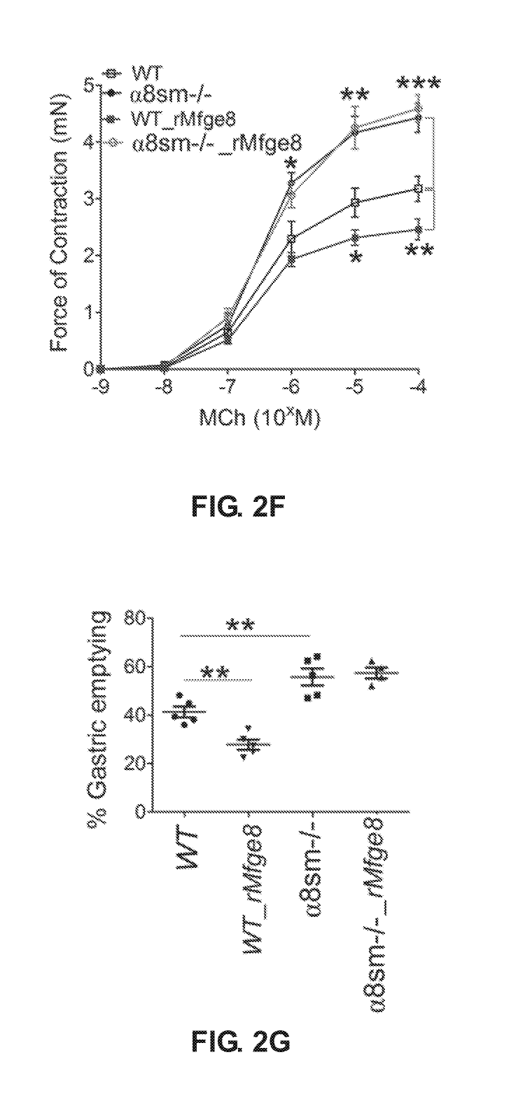

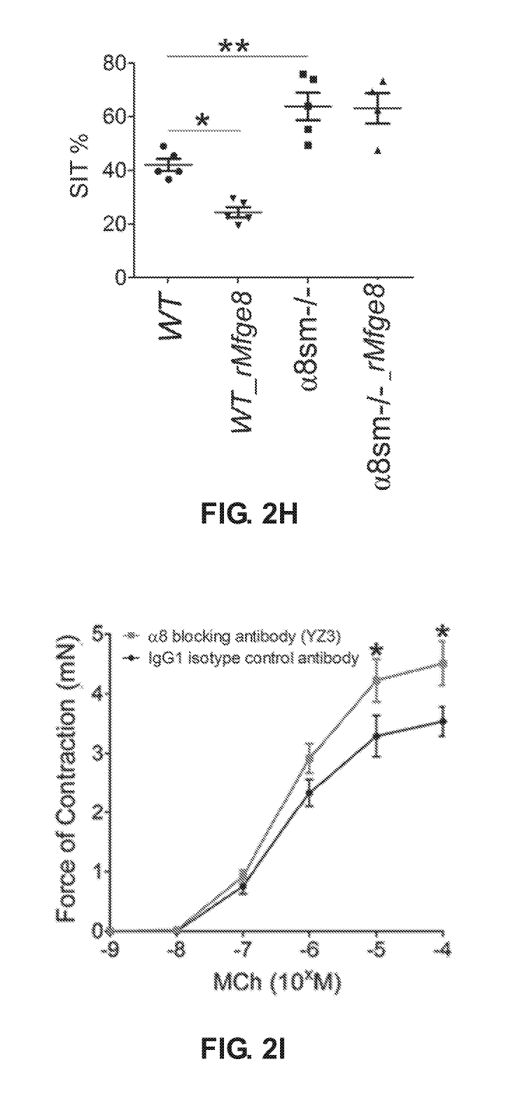

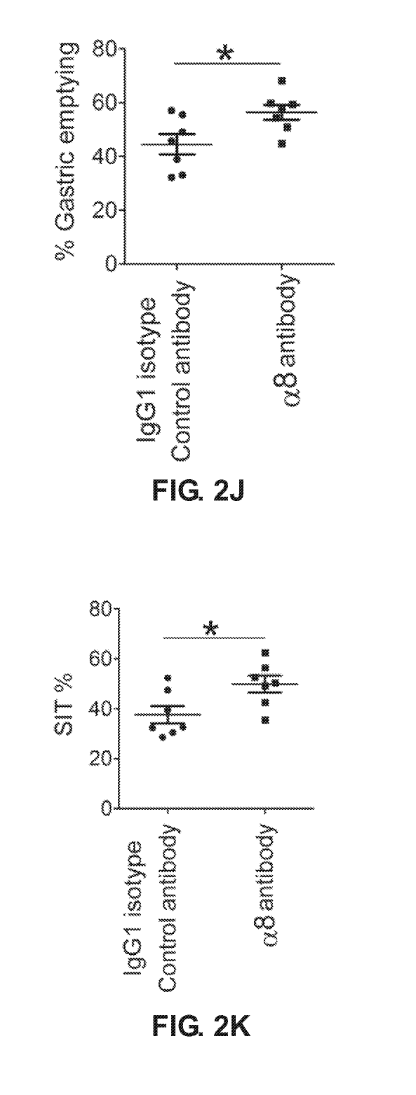

We have previously shown that Mfge8 is not a ligand for the RGD-binding integrins αᵥβ₆, αᵥβ₈, and α₅β₁⁸, leaving the α₈β₁ and αᵥβ₁ as the potential RGD binding receptors for the effect of Mfge8 on smooth muscle contraction. We initially focused on the α₈β₁ because of its high expression in smooth muscle^{17, 32}. To determine if α8β1 is a receptor for Mfge8, we used a solid-phase assay to analyze the direct binding of Mfge8 to purified α8. We included purified αvβ3 and α5β1 as positive and negative controls, respectively. Mfge8 bound to α8β1 and αvβ3, but not to α5β1 (FIG. 2A). To further confirm this interaction, we evaluated cell adhesion of SW480 cells, a human colon cancer cell line, transfected with α8 or β3 to Mfge8 (FIG. 2B). Control SW480 cells express the Mfge8 ligand αvβ5 as well as α5β1 and bind Mfge8 in an αvβ5-dependent manner. We first compared adhesion of α8-transfected cells with adhesion of β3-transfected cells expressing αvβ3, a known receptor for Mfge8 (FIG. 2B), to Mfge8. Mock-transfected SW480 cells adhered to Mfge8 and adherence was blocked by anti-β5 antibody (ALULA). In the presence of ALULA, β3-transfected cells adhered to Mfge8, and adherence was blocked by an anti-β3 antibody (LM609). α8-transfected SW480 cells adhered to Mfge8 in the presence of ALULA, and adherence was blocked by the addition of α8 blocking antibody (YZ83). These results indicate that α8β1 specifically mediates cell adhesion to Mfge8. As a positive control for this assay, we assessed adhesion of β3- and α8-transfectant to tenascin-C, a known common ligand to αvβ3 and α8β1, and inhibition by the anti-β3 (LM609) and the anti-α8 (YZ83) blocking antibodies (FIG. 2B). Next we analyzed adhesion of α8-transfected SW480 cells to Mfge8 at various concentrations in the presence of ALULA (FIG. 2C). The α8-transfected cells adhered to Mfge8 in a dose-dependent fashion.

To confirm these findings in smooth muscle cells, we evaluated adhesion of primary human gastric smooth muscle cells to Mfge8. Primary human gastric smooth muscle cells expressed the β₅, β₁, αᵥ, and α₈ integrin subunits and adhered to Mfge8 (FIGS. 2D and 2E). Adherence was significantly inhibited by blocking antibodies to the β₅, β₁, αᵥ, and α₈ subunits but not the α₅ integrin. Simultaneously blocking both the αᵥ and as integrins had a significantly greater effect on adhesion than blocking each integrin individually (FIG. 2E).

### Example 3—α8β1 Mediates the Effect of Mfge8 on Motility

To evaluate whether α₈β₁ mediates the effect of Mfge8 on gastric smooth muscle, we created a transgenic mouse line containing α₈ floxed/floxed alleles, a tetracycline-inducible Cre construct, and then α-smooth muscle-rtTA construct (α₈sm^{-/-}). The addition of doxycycline chow resulted in smooth muscle specific deletion of α₈. Gastric antral smooth muscle from α₈sm^{-/-} had enhanced contraction in response to MCh and KCl (FIG. 2F) and enhanced calcium sensitivity as assessed by increased phosphorylation of MYPT and MLC and enhanced RhoA activation. Unlike WT samples, rMfge8 did not significantly reduce the force of contraction, rescue enhanced calcium sensitivity, or reduce RhoA activation in α₈sm^{-/-} antral smooth muscle. α₈sm^{-/-} mice had enhanced gastric emptying and SIT (FIGS. 2G and 2H). Oral gavage with rMfge8 did not significantly slow gastric emptying or small intestinal transit times in α₈sm^{-/-} mice (FIGS. 2G and 2H).

Administration of an α8 blocking antibody to WT mice significantly increased the force of antral contraction, accelerated gastric emptying and reduced SIT (FIGS. 2I, 2J, and 2K). The antibody used was described in U.S. Pat. No. 8,658,770, incorporated herein by reference in its entirety. In sum, these data indicate that disruption of α8β₁ integrin signaling accelerates gastrointestinal motility.

### Example 4—α8β₁ Integrin Inhibits PI3 Kinase

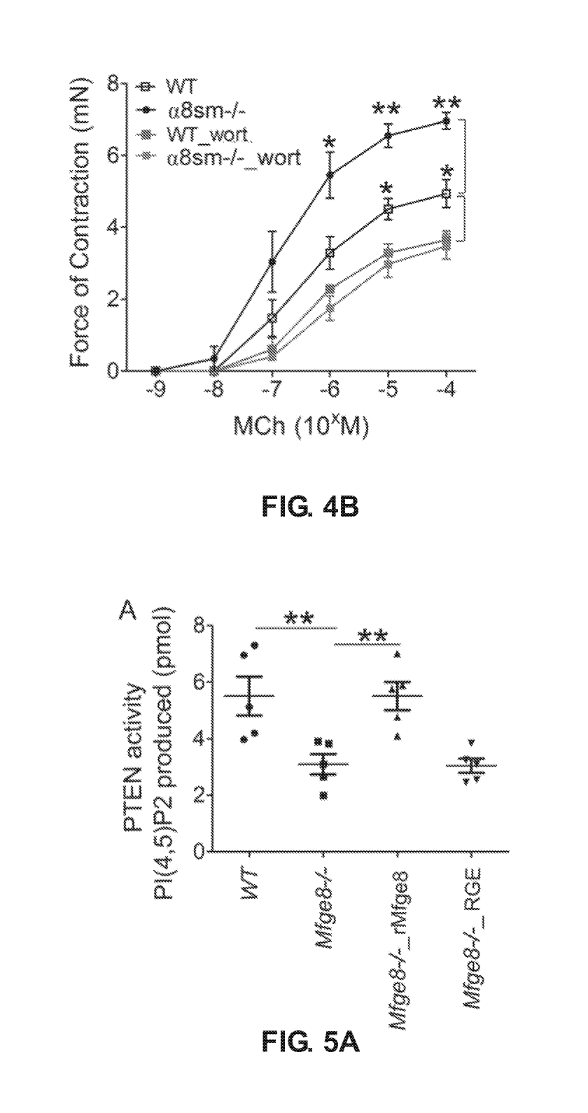

PI3 kinase (PI3K) is a positive regulator of smooth muscle contraction. To determine whether Mfge8 modulates smooth muscle contraction through PI3K, we incubated antral smooth muscle rings with the PI3K inhibitor wortmannin. Wortmannin significantly reduced contraction in Mfge8^{-/-}, α8sm^{-/-}, and WT antral smooth with a proportionally greater effect in antrum from Mfge8^{-/-} and α8sm^{-/-} as compared with antrum from WT mice (FIGS. 4A and 4B). PI3K activation leads to phosphorylation of AKT. Antral rings from Mfge8^{-/-} and αsm^{-/-} mice had enhanced phosphorylation of AKT at serine 473. rMfge8 reduced AKT phosphorylation in Mfge8^{-/-} but not α8sm^{-/-} samples. Wortmannin also prevented the enhanced RhoA activation in Mfge8^{-/-} and α8sm^{-/-} antral smooth muscle.

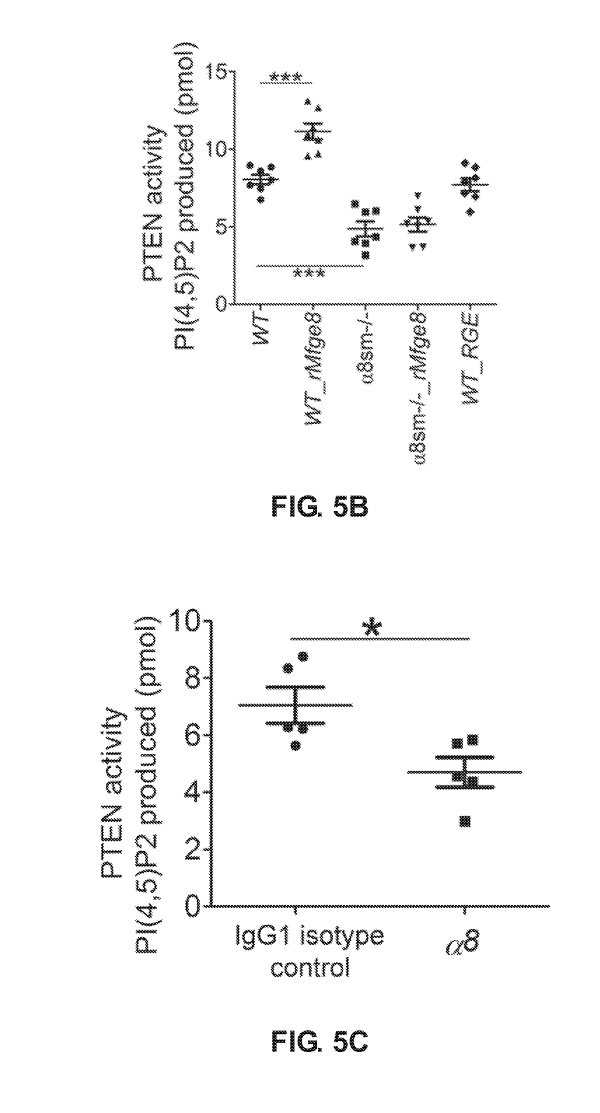

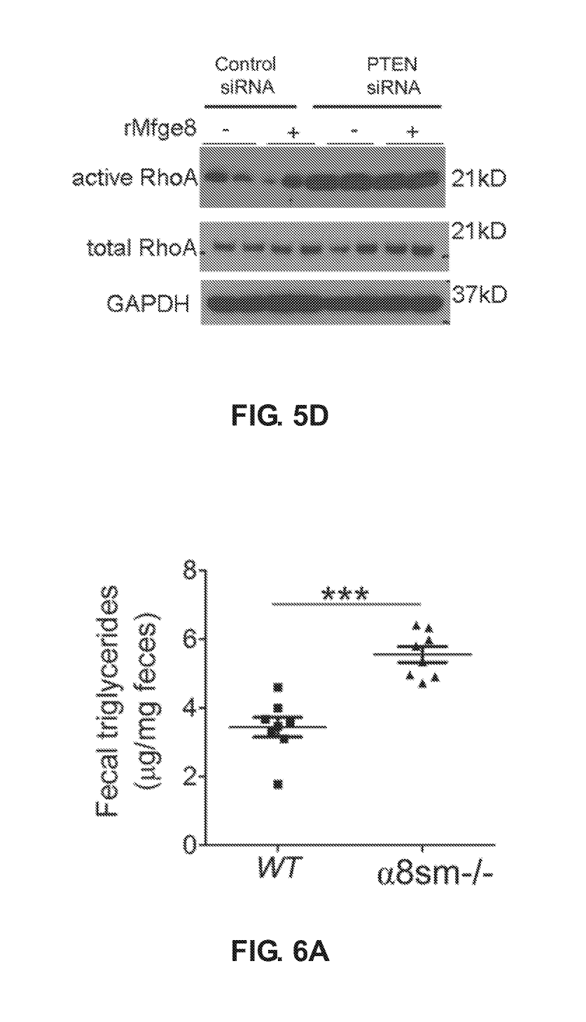

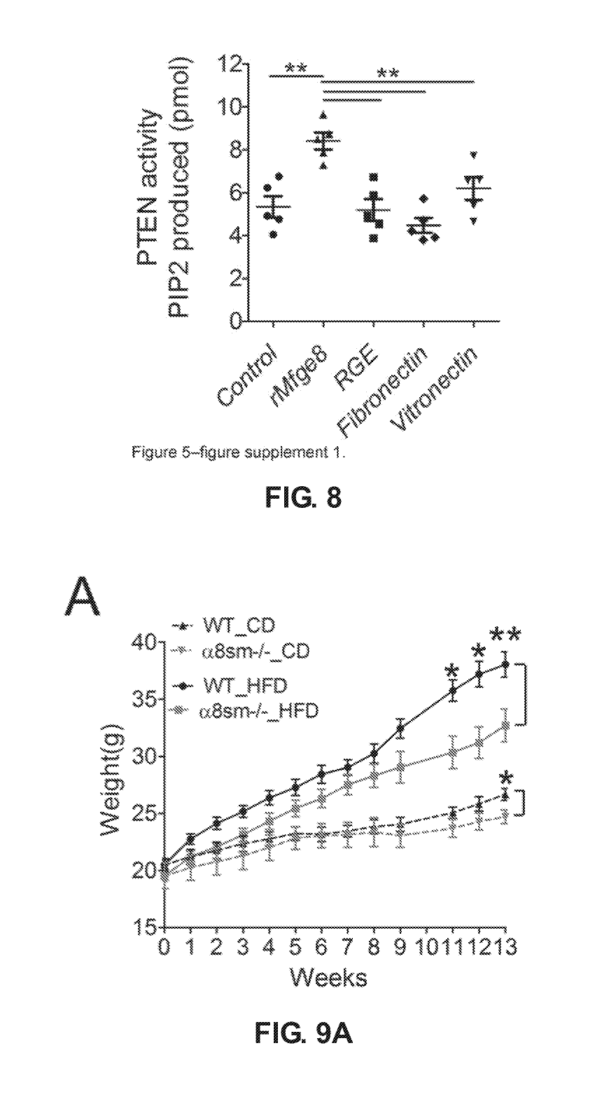

Phosphatase and tensin homolog (PTEN) is the major negative regulator of PI3K³⁶. To determine whether Mfge8 ligation of α8β1 opposed PI3K activation through PTEN, we measured PTEN activity using an ELISA that measures PIP2 production. PTEN activity was reduced in both Mfge8^{-/-} and α8sm^{-/-} antral rings (FIGS. 5A and 5B). rMfge8 significantly increased PTEN activity in antrum from WT and Mfge8^{-/-} mice with no effect in antrum from α8sm^{-/-} mice (FIGS. 5A and 5B). In WT mice there was a significant inverse correlation between the extent of PTEN activity and the rate of gastric emptying and small intestinal transit time. rMfge8 increased PTEN activity in primary human gastric smooth muscle cells, an effect that was blocked by blocking antibody to α8 but not to α5 or β5 integrin subunits (FIG. 5C). Of note, treatment with fibronectin or vitronectin, both ligands of α₈β₁, did not increase PTEN activity, suggesting a specific effect for Mfge8 (FIG. 8). We next used siRNA to knock down PTEN expression in primary human gastric smooth muscle cells and to evaluate the effect on smooth muscle calcium sensitivity. PTEN knockdown led to increased MLC and MYPT phosphorylation in response to 5-HT as well as to increased RhoA activation (FIG. 5D). Unlike control samples, rMfge8 did not reduce the degree of MYPT or MLC phosphorylation or RhoA activation in gastric smooth muscle after PTEN knockdown (FIG. 5D). These data indicate that α8β1 prevents RhoA activation in gastric smooth muscle by increasing the activity of PTEN.

### Example 5—α8β₁ Integrin Promotes Nutrient Absorption

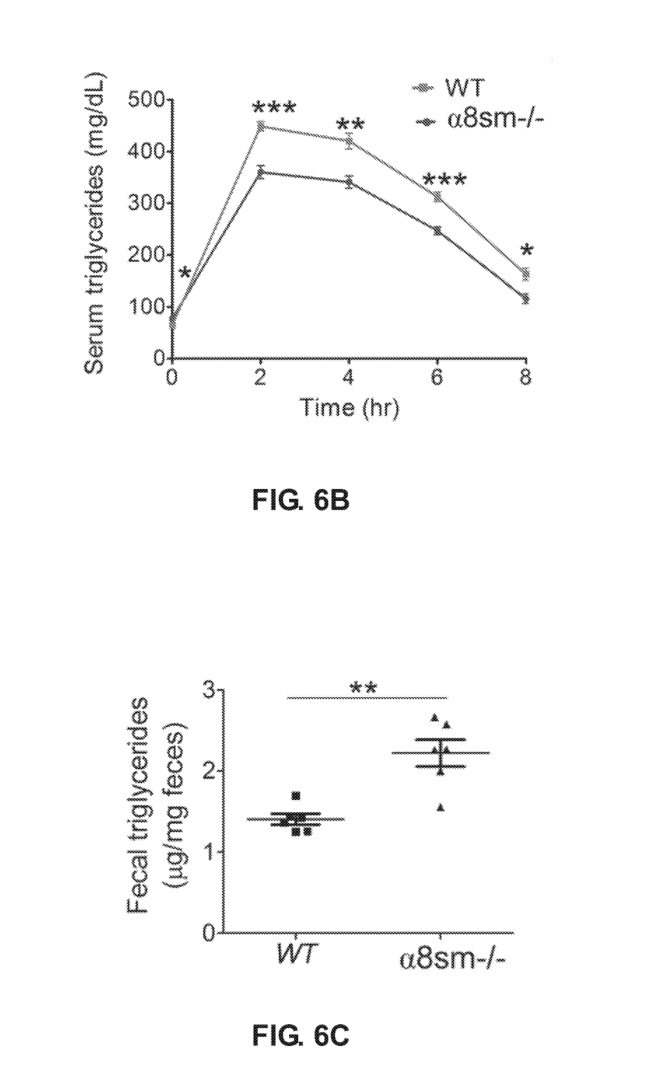

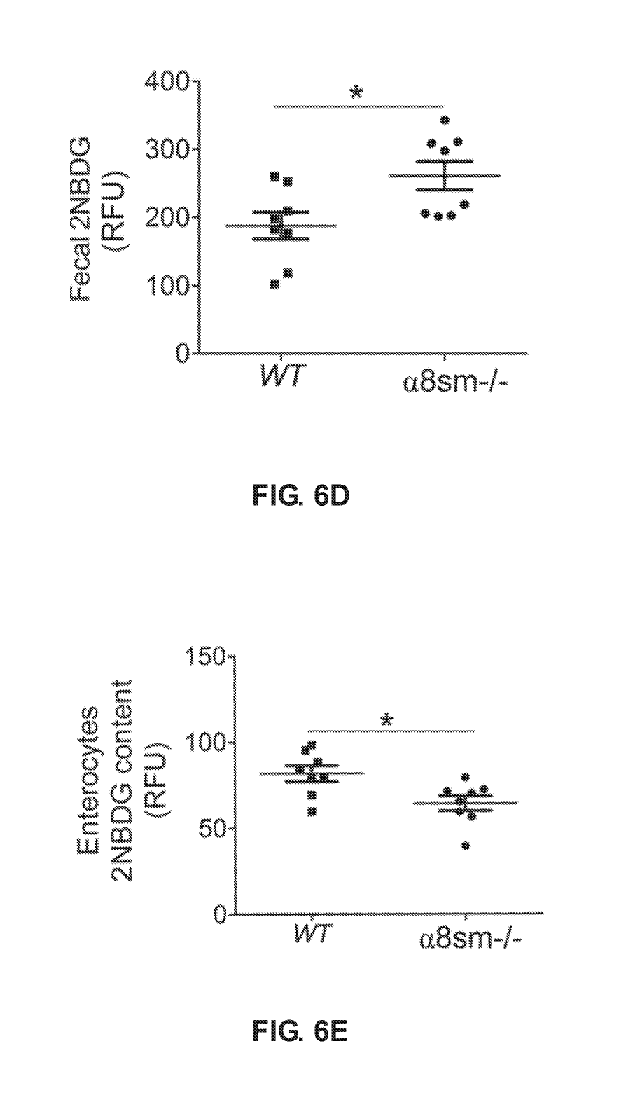

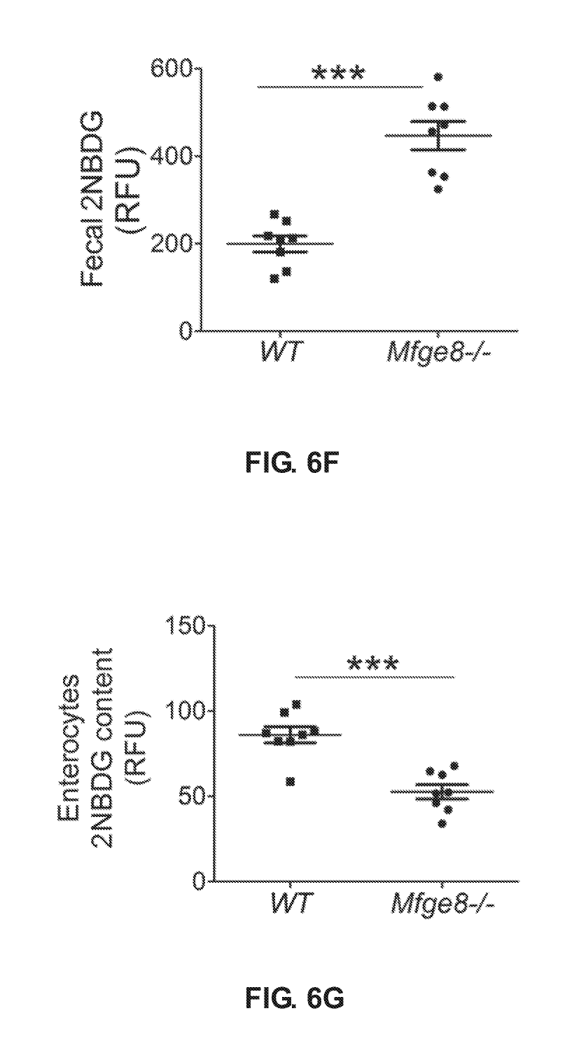

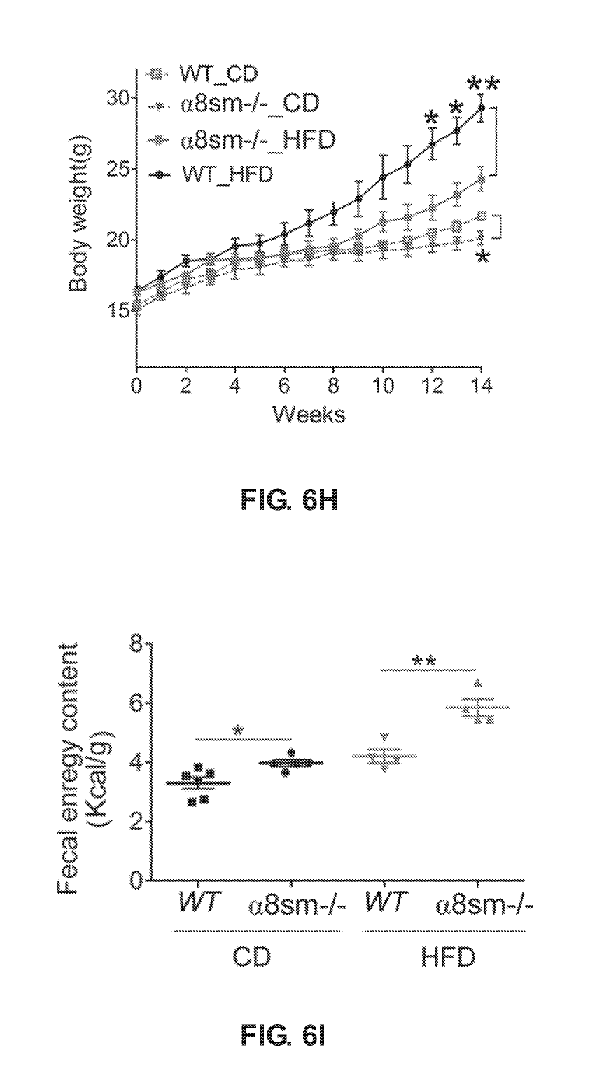

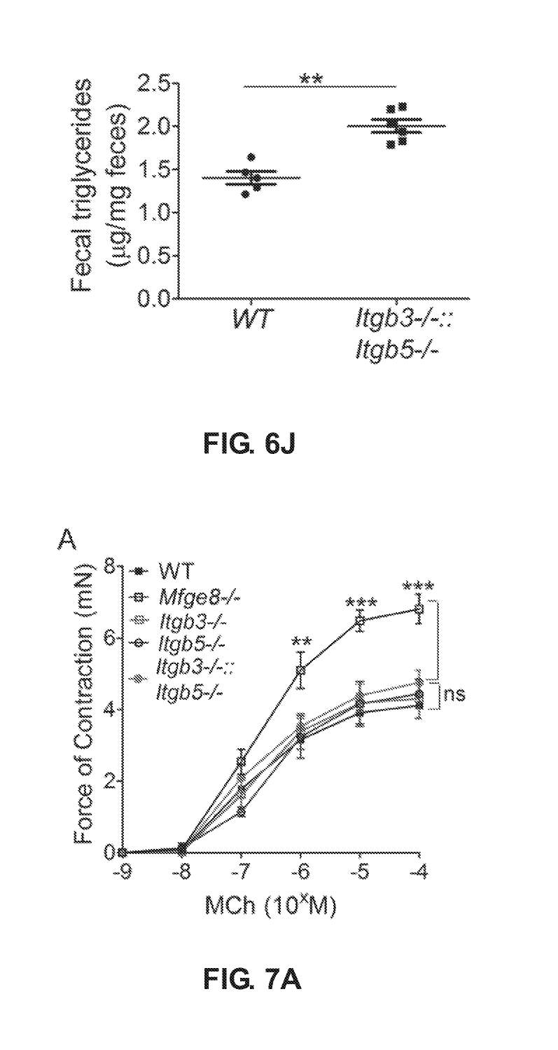

We next wanted to evaluate the functional consequences of altered motility on nutrient absorption in α8sm^{-/-} mice. Since we have previously reported impaired fat absorption in Mfge8^{-/-} mice, we first assessed the ability of α8sm^{-/-} mice to absorb dietary fats. After an olive oil gavage, α8sm^{-/-} mice had significantly higher fecal triglyceride (TG) concentrations (FIG. 6A) as well as lower serum TG levels (FIG. 6B) as compared with WT control mice. Fecal TG levels were also significantly higher in mice on a normal chow diet (NCD) as compared with WT mice (FIG. 6C). Of note, primary enterocytes isolated from α8sm^{-/-} mice did not have a defect in fatty acid uptake indicating that the increase in stool fat was not due to a defect in enterocyte fatty acid uptake. Furthermore, IP injection of olive oil resulted in similar serum TG levels in α8sm^{-/-} mice as compared with WT mice indicating that clearance of lipids by tissue outside of the intestinal tract was preserved in α8sm^{-/-} mice. Taken together, these data indicate that α8sm^{-/-} mice develop steatorrhea.

To evaluate whether malabsorption was specific for fat or represented a more generalized impairment of nutrient uptake, we measured stool glucose levels after gavage with a 2-(N-(7-Nitrobenz-2-oxa-1,3-diazol-4-yl)Amino)-2-Deoxyglucose (2NBDG) fluorescent glucose analog mixed with methylcellulose, to form a semisolid bolus. α8sm^{-/-} mice had increased stool glucose levels (FIG. 6D) coupled with reduced enterocyte glucose levels (FIG. 6E). Enterocytes isolated from α8sm^{-/-} mice did not have a defect in 2NBDG uptake in vitro. Mfge8^{-/-} mice also had increased stool 2NBDG and reduced enterocyte 2NBDG levels (FIGS. 6F and 6G) when 2NBDG was gavaged as a semisolid mixed with methylcellulose, but not when administered as a liquid preparation in PBS.

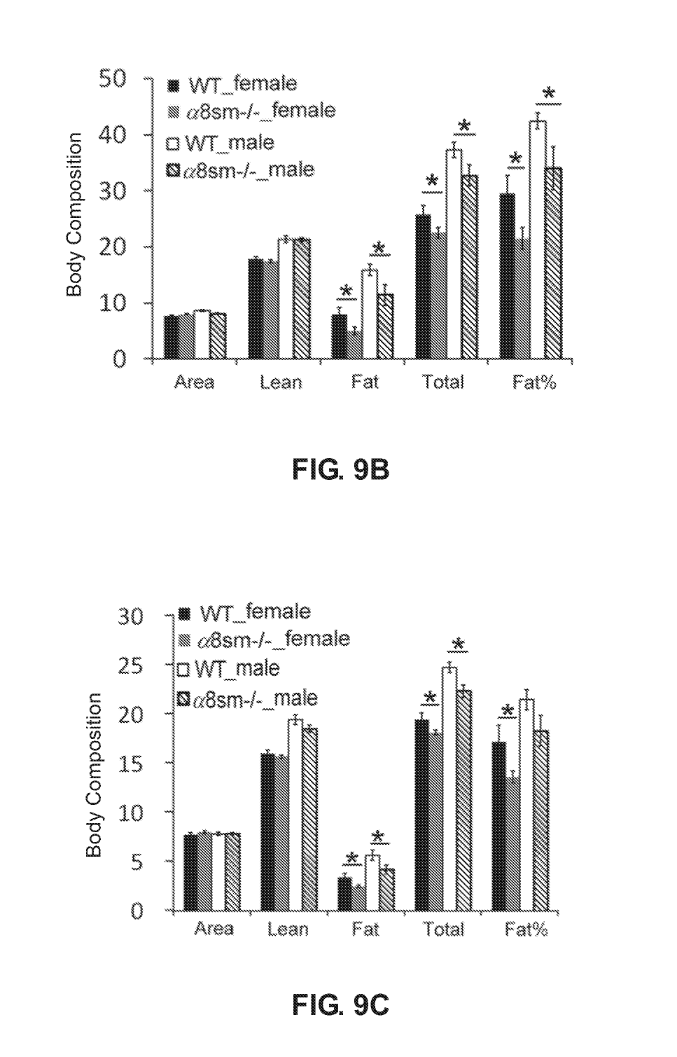

Mfge8^{-/-} mice gain approximately 50% less weight on a high-fat diet (HFD) as compared with WT controls. To evaluate the relative contribution of altered motility to this phenotype, we placed α8sm^{-/-} mice on a HFD. Both female and male α₈sm^{-/-} mice were significantly protected from weight gain on a HFD (FIG. 6H). Reduced weight gain on a HFD in α₈sm^{-/-} mice was associated with reduced body fat as measured by Dexa scanning (FIGS. 9B and 9C). A modest reduction in body weight was also apparent in α8sm^{-/-} mice on a NCD as compared with WT controls and became statistically significant at 22 weeks of age and was associated with decreased body fat on DEXA scan (FIG. 9C). α₈sm^{-/-} mice also had increased stool energy content as measured by bomb calorimetry on both a HFD and NCD (FIG. 6I).

### Materials and Methods

Mice.

All animal experiments were approved by the UCSF Institutional Animal Care and Use Committee in adherence to NIH guidelines and policies. All mice were maintained on a C57BL/6J background. Mfge8^{-/-} mice were obtained from RIKEN. (tetO)7-Cre and α-sm-rTTA mouse lines have been described previously. Mfge8^{-/-}sm⁺ transgenic mice were created by cloning the Mfge8 long isoform into the PTRE2 vector with subsequent microinjection of DNA by the Gladstone Institute Gene-Targeting Core. Transgenic mice containing the tetracycline-inducible Mfge8 construct were crossed with a Mfge8^{-/-} mice line created using a gene disruption vector and mice carrying the (tetO)7-Cre and α-sm-rTTA transgenes. α₈ floxed mice of been previously described. α8sm^{-/-} mice were created by crossing α₈ floxed mice with mice carrying the (tetO)7-Cre and α-sm-rTTA transgenes. β3−/− and δ5−/− mice in the 129 SVEV strain have been previously described. For smooth muscle induction of Mfge8 or Cre-mediated recombination of α₈. Mice were placed on doxycycline chow for 2 weeks prior to experiments.

Antral Ring Contraction.

We suspended freshly isolated antral ring slices (2-3 mm in length) on plexiglass rods in a double-jacketed organ bath (Radnoti 8 unit tissue organ bath system) in Krebs-Henseleit solution maintained at 5% CO2-95% O2, 37° C., and a pH of 7.4-7.4533. We attached rings by a silk thread to aFT03 isometric transducer. Concentration response curves of multiple chambers were continuously displayed and recorded. We set initial tension at 0.5 g for antral rings before adding contractile agonists. We then added a range of concentrations of MCh (10⁻⁴ to 10⁻⁹M) and KCl (3.75-60 mM) to induce contraction. For selected studies, wortmannin (100 ng/ML), Y-27632 (100 nm) or recombinant Mfge8 constructs (10 μg/ml) were added 15 minutes prior to addition of contractile agonists

Gastric Emptying Measurement.

Mice were deprived of food for 12 h prior to experimentation but had free access to water. Mice were gavage with 250 μl of methylcellulose mixed with phenol red (0.5 g/L phenol red in 0.9% NaCl with 1.5% methylcellulose). We euthanized mice 15 minutes after administration of the test meal, dissected out the stomach and removed the abdomen after ligation of the cardiac and pyloric ends to ensure that any retained meal did not leak out of the stomach during removal. We then cut the stomach into pieces and homogenized with 25 ml of 0.1 N NaOH and added 0.5 ml of trichloroacetic acid (20% w/v) and centrifuged at 3000 rpm for 20 minutes. We then added 4 ml of 0.5 N NaOH to 1 ml of the supernatant and measured absorbance at 560 nm to assess phenol red content in the stomach. The percentage gastric emptying was derived as (1−X/Y)*100 where X represents absorbance of phenol red recovered from the stomach of animals sacrificed 15 minutes after test meal. Y represents mean (n=5) absorbance of phenol red recovered from the stomachs of control animals which were euthanized 0 min following the test meal. In experiments using rMfge8 and RGE constructs, we administered each construct by gavage (50 μg/kg body weight in a total volume of 200 μl in PBS) before administration of phenol to mice. Y-27632 was administered IP (100 nm) 15 minutes prior to gavage.

Small Intestinal Transit (SIT).

We deprived mice of food for 12 h prior to experimentation while allowing free access to water. We then gavaged mice with 250 μl Carmine meal (6% Carmine red and 0.5% methylcellulose in water). 15 minutes after administration of gavage, we euthanized mice and dissected out the small intestine from the pylorus to the ileocecal junction, identifying the location to which the meal had traversed, and securing that position with thread to avoid changes in the length of the transit due to handling. The small intestinal transit (SIT) was calculated from the distance traveled by Carmine meal divided by total length of the small intestine multiplied by 100. In experiments using rMfge8 and RGE constructs, we administered each construct by gavage (50 μg/kg body weight in a total volume of 200 μl in PBS) before administration of the Carmine meal to mice. Y-27632 was administered IP (100 nm) 15 minutes prior to gavage.

Primary Enterocytes Isolation.

We collected primary enterocytes by harvesting the proximal small intestine from anesthetized mice, emptying the luminal contents, washing with 115 mM NaCl, 5.4 mM KCl, 0.96 mM NaH2PO4, 26.19 mM NaHCO3 and 5.5 mM glucose buffer at pH 7.4 and gassing for 30 min with 95% O2 and 5% CO2. We then filled the proximal small intestines with buffer containing 67.5 mM NaCl, 1.5 mM KCl, 0.96 mM NaH2PO4, 26.19 mM NaHCO3, 27 mM sodium citrate and 5.5 mM glucose at pH 7.4, saturated with 95% O2 and 5% CO2, and incubated in a bath containing oxygenated saline at 37° C. with constant shaking. After 15 min, we discarded the luminal solutions and filled the intestines with buffer containing 115 mM NaCl, 5.4 mM KCl, 0.96 mM NaH2PO4, 26.19 mM NaHCO3, 1.5 mM EDTA, 0.5 mM dithiothreitol and 5.5 mM glucose at pH 7.4, saturated with 95% O2 and 5% CO2, and we placed them in saline as described above. After 15 min, we collected and centrifuged the luminal contents (1,500 r.p.m., 5 min, room temperature) and resuspended the pellets in DMEM saturated with 95% O2 and 5% CO2).

Olive Oil/2NDGB Gavage.

We fasted 6- to 8-week-old mice for 4 h and then each mouse received an oral gavage of 200 μl olive oil or 2 μg per g body weight 2NBDG and 2 μg per g body weight rhodamine-PEG (Methoxyl PEG Rhodamine B, MW 5.000 g mol⁻¹) with 0.2% fatty acid-free BSA by gavage. We collected feces from 20 min to 4 h after 2NBDG was administered. We homogenized 50 mg of feces in PBS containing 30 mM HEPES, 57.51 mM MgCl2 and 0.57 mg ml⁻¹ BSA with 0.5% SDS and sonicated for 30 s; we then centrifuged at 1,000 g for 10 min. We transferred supernatants to 96-well plates and measured fluorescence values immediately using a fluorescence microplate reader for endpoint reading (Molecular Devices). We then subtracted baseline fluorescence from untreated mice from measured fluorescence. We also measured enterocytes' 2NBDG content after isolation of primary cells as described above, using excitation and emission wavelengths of 488 nm and 515 nm, respectively. For rhodamine-PEG, the excitation and emission wavelengths were 575 nm and 595 nm, respectively.

Solid Phase Binding Assay.

Direct binding of Mfge8 with α8 was assessed by solid-phase binding in non-tissue coated microplates. Either recombinant α8, αvβ3, or α5β1 were attached to the plates and purified Mfge8 was added for 2 h at room temperature in the presence or absence of 10 mM EDTA. For α5β1, 1 mM MgCl²⁺ and 1 mg/mL CaCl²⁺ was added to activate β1. Following 5 washes with PBS+1% BSA and 0.05% Tween, the extent of Mfge8 binding was detected using a biotinylated antibody against Mfge8 (1:1000, 1 h at 37 C). Then streptavidin-HRP was added for 20 min at room temperature followed by 3,3′,5,5′ tetramethylbenzidine substrate solution. Absorbance was then measured at 450 nm in a microplate reader.

Serum and Fecal Triglycerides Measurement.

We fasted 6-8 week old mice for 4 h and administered 200 μl olive oil by oral gavage or IP injection, and collected tail vein blood at indicated times. Serum TG concentration was determined by Wako L-Type TG determination kit (Wako Chemicals USA). We collected the feces from 20 min to 4 h after Olive oil administration. 50 mg of feces were homogenized with chloroform/methanol (2:1) in a 20:1 v/w ratio, the whole mixture was incubated overnight at 4° C. with gentle shaking. Then, 0.2 volume of 0.9% NaCl was added and centrifuged at 500 g for 30 minutes After extracting the organic phase, samples were evaporated under nitrogen until dry and reconstituted in PBS containing 1% Triton X-100 for TG measurement by Wako L-Type TG determination kit (Wako Chemicals USA).

Cell Adhesion Assay.

Cell adhesion assays were performed as described⁴⁵ with slight modifications. Briefly, 1×10⁵ cells were seeded into each well of 96 well MaxiSorp enzyme-linked immunosorbent assay plates (Nunc) coated with substrate proteins at 37° C. for 1 h and then incubated for 1 h at 37° C. Attached cells were stained with 0.5% crystal violet and solubilized in 2% Tri-ton X-100 for taking optical density at 595 nm. For blocking experiments, cells were incubated with antibodies before plating for 15 minutes on ice.

Human Gastric Smooth Muscle Cells (HGSMCs)/siRNA Treatment.

HGSMCs were obtained from commercial sources (Science Cell Research Laboratories) and maintained in minimum essential medium supplemented with 10% FBS at 37° C. with 5% CO2. We plated the cells in six-well plates 1 day prior to infection. We transfected cells with 100 nM PTEN siRNA (ON-TARGETplus Human PTEN, Thermo Fisher Scientific) or controls (ON-TARGETplus Scramble Control siRNA, Human, Thermo Fisher Scientific) in antibiotic- and norepinephrine-free culture medium using Lipofectamine-2000 (Invitrogen). 6 hours later, we change the medium to fully supplemented medium and conducted assays 48 h after transfection.

RhoA Activation Assay.

The RhoA activation assay was performed according to the manufacturer's instructions (Cytoskeleton). Briefly, we dissected out the gastric antrum, gently removed the mucosal layer and homogenized the muscle layer in lysis buffer (50 mM Tris-HCl, pH 7.5, 10 mM MgCl2, 0.5 M NaCl, 1% Triton X-100, and protease and phosphatase inhibitor cocktail (Thermo)). We collected the supernatants after centrifugation and incubated with GST-Rhotekin bound to glutathione-agarose beads at 4° C. for 1 h. We washed the beads with a wash buffer containing 25 mM Tris, pH 7.5, 30 mM MgCl2, and 40 mM NaCl. GTP-bound RhoA was detected by immunoblotting.

PTEN Activity Assay.

We isolated antral lysates or human gastric smooth muscle cell lysates and measured conversion of PIP3 to PIP2 (PTEN activity ELISA, Echelon) after incubation with recombinant proteins (rMfge8 or RGE 10 ug/ml) or blocking antibodies against α8, α5 and β5 (10 ug/ml). We incubated lysates on a PI(4,5)P2 coated microplate and added a PI(4,5) P2 detector protein. We used a peroxidase-linked secondary detector to detect PI (4, 5) P2 detector binding to the plate in a colorimetric assay where the colorimetric signal is inversely proportional to the amount of PI (4, 5) P2 produced by PTEN.

Western Blots.

We lysed tissues in cold RIPA buffer (50 mM Tris HCl pH 7.5, 150 mM NaCl, 1% NP-40, 1% sodium deoxycholate, 0.1% SDS) supplemented with complete miniprotease and phosphatase inhibitor cocktail (Pierce, Rockford, Ill.). We incubated lysates at 4° C. with gentle rocking for 30 min, sonicated on ice for 30 seconds (in 5 second bursts) and then centrifuged at 12,800 rpm for 15 min at 4° C. We determined protein concentration by Bradford assay (Bio-Rad. Hercules, Calif.). We separated 20 μg of protein by SDS-PAGE on 7.5% resolving gels (Bio-Rad) and transblotted onto polyvinylidene fluoride membranes (Millipore). We incubated the membranes with a 1:1,000 dilution of antibodies against Akt (catalog 9272, Cell Signaling), phospho-Akt Scr473 (clone 193H12, Cell Signaling), MLC (catalog 3672S Cell Signaling) phospho-MLC (clone 519, Cell Signaling), MYPT (clone, Cell Signaling) phospho-MYPT (catalog 5163, Cell Signaling). RhoA (clone 67139, Cell Signaling), PTEN (clone 138G6, Cell Signaling), or GAPDH (clone 14C10, Cell Signaling) followed by a secondary HRP-conjugated antibody. For evaluation of total Akt, MLC or MYPT we stripped and reprobed membranes that been blotted for phospho-versions of these proteins. Blots were developed using enhance chemical luminescence system (Amersham).

Recombinant Protein Production.

We created and expressed recombinant Mfge8 and RGE protein constructs in High Five cells as previously described. All constructs were expressed with a human Fc domain for purification across a protein G sepharose column. For experiments in FIGS. 3A and 3B, Mfge8 was expressed in Freestyle 293 cells with His-tag and purified by Ni-NTA column. Third fibronectin III repeat of tenascin-C (TNfn3) was prepared as described.

High-Fat Diet.

We placed 8-week-old α8sm^{-/-} mice on a high-fat diet formula containing 60% fat calories (Research Diets) for 12 weeks. Mouse were placed on doxycycline chow (2 g/kg, Bioserve) for two weeks prior to beginning the HFD and subsequently had doxycycline in their water (0.2 mg/ml) for the duration of the experiments.

Body Composition Analysis.

We performed bone, lean and fat mass analysis with a GE Lunar PIXImus II Dual Energy X-ray Absorptiometer.

Measurements of Fecal Energy Content.

We freeze-dried feces from mice on a HFD and pulverized them with a ceramic mortar and pestle. We measured caloric content of feces with an 1108 Oxygen Combustion Bomb calorimeter.

Statistical Analyses.

We assessed data for normal distribution and similar variance between groups using GraphPad Prism 6.0. We used a one-way ANOVA to make comparisons between multiple groups. When the ANOVA comparison was statistically significant (P<0.05), we performed further pairwise analysis using a Bonferroni t-test. We used a two-sided Student's t-test for comparisons between 2 groups. For analysis of weight gain over time in mice, we used a two-way ANOVA for repeated measures. We used GraphPad Prism 6.0 for all statistical analyses. We presented all data as mean±s.e.m. We selected sample size for animal experiments based on numbers typically used in the literature. We did not perform randomization of animals.

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

1. An isolated monoclonal antibody, wherein the antibody specifically binds to an integrin α8β1 receptor and wherein the antibody competes for binding of the receptor with Milk fat Globule Epidermal Growth Factor like 8 (Mfge8).

2. The isolated antibody of any one of claim 1, wherein the antibody is recombinant.

3. The isolated antibody of claim 1, wherein the antibody is an IgG, IgM, IgA or an antigen binding fragment thereof.

4. The isolated antibody of claim 1, wherein the antibody is a Fab′, a F(ab′)2, a F(ab′)3, a monovalent scFv, a bivalent scFv, or a single domain antibody.

5. The isolated antibody of claim 1, wherein the antibody is a human, humanized, or de-immunized antibody.

6. The isolated antibody of claim 1, wherein the antibody binds with a high affinity to a protein having at least a 90% sequence identity to SEQ ID NO: 1.

7. The isolated antibody of claim 6, wherein the antibody binds with a high affinity to a protein having the sequence of SEQ ID NO: 1.

8. A composition comprising an antibody of claim 1 in a pharmaceutically acceptable carrier.

9. An isolated polynucleotide molecule comprising a nucleic acid sequence encoding a protein incorporated into an antibody of claim 1.

10. A method for treating a gastrointestinal motility disorder in a patient comprising administering to the patient an antibody that disrupts the α8β1/Mfge8 interaction in an amount effective to treat the gastrointestinal motility disorder.

11. A method for treating a subject having a gastrointestinal motility disorder comprising administering an effective amount of an antibody of claim 1 to the subject.

12. The method of claim 11, wherein the gastrointestinal motility disorder is diabetic gastropathy, idiopathic gastroparesis, opioid-induced constipation, drug-induced ileus, idiopathic chronic constipation, intestinal pseudo-obstruction, bowel hypomotility, functional bowel disorders, constipation-predominant Irritable Bowel Syndrome, gastrointestinal-dysmotility, or obesity.

13. The method of claim 12, wherein the diabetic gastropathy is idiopathic gastroparesis.

14. The method of claim 11, wherein the antibody is administered systemically.

15. The method of claim 11, wherein the antibody is administered intravenously, intradermally, intramuscularly, intraperitoneally, subcutaneously, anally or orally.

16. The method of claim 11, further comprising administering at least a second gastrointestinal motility disorder therapy to the subject.

17. The method of claim 16, wherein the second gastrointestinal motility disorder therapy enhances the therapeutic or protective effect, and/or increases the therapeutic effect of antibody that disrupts the α8β1/Mfge8 interaction.

18. A composition comprising an α8β1 binding antibody, for use in the treatment of a gastrointestinal motility disorder in a patient.

19. The composition according to claim 18, wherein the antibody is a monoclonal antibody, a polyclonal antibody, a chimeric antibody, an affinity matured antibody, a humanized antibody, a human antibody, or an antigen-binding antibody fragment.

20.-29. (canceled)


SEQUENCE LISTING (5 sequences)

SEQ ID NO: 1 - protein, 1063 aa, Homo sapiens
MSPGASRGPRGSQAPLIAPLCCAAAALGMLLWSPACQAFNLDVEKLTVYSGPKGSYFGYAVDFHIPDARTASVLVGAPKANTSQPDIVEGGAVYYCPWPAEGSAQCRQIPFDTTNNRKIRVNGTKEPIEFKSNQWFGATVKAHKGKVVACAPLYHWRTLKPTPEKDPVGTCYVAIQNFSAYAEFSPCRNSNADPEGQGYCQAGFSLDFYKNGDLIVGGPGSFYWQGQVITASVADIIANYSFKDILRKLAGEKQTEVAPASYDDSYLGYSVAAGEFTGDSQQELVAGIPRGAQNFGYVSIINSTDMTFIQNFTGEQMASYFGYTVVVSDVNSDGLDDVLVGAPLFMEREFESNPREVGQIYLYLQVSSLLFRDPQILTGTETFGRFGSAMAHLGDLNQDGYNDIAIGVPFAGKDQRGKVLIYNGNKDGLNTKPSQVLQGVWASHAVPSGFGFTLRGDSDIDKNDYPDLIVGAFGTGKVAVYRARPVVTVDAQLLLHPMIINLENKTCQVPDSMTSAACFSLRVCASVTGQSIANTIVLMAEVQLDSLKQKGAIKRTLFLDNHQAHRVFPLVIKRQKSHQCQDFIVYLRDETEFRDKLSPINISLNYSLDESTFKEGLEVKPILNYYRENIVSEQAHILVDCGEDNLCVPDLKLSARPDKHQVIIGDENHLMLIINARNEGEGAYEAELFVMIPEEADYVGIERNNKGFRPLSCEYKMENVTRMVVCDLGNPMVSGTNYSLGLRFAVPRLEKTNMSINFDLQIRSSNKDNPDSNFVSLQINITAVAQVEIRGVSHPPQIVLPIHNWEPEEEPHKEEEVGPLVEHIYELHNIGPSTISDTILEVGWPFSARDEFLLYIFHIQTLGPLQCQPNPNINPQDIKPAASPEDTPELSAFLRNSTIPHLVRKRDVHVVEFHRQSPAKILNCTNIECLQISCAVGRLEGGESAVLKVRSRLWAHTFLQRKNDPYALASLVSFEVKKMPYTDQPAKLPEGSIVIKTSVIWATPNVSFSIPLWVIILAILLGLLVLAILTLALWKCGFFDRARPPQEDMTDREQLTNDKTPEA

SEQ ID NO: 2 - protein, 1062 aa, Mus musculus
MSAGTHCGPPGNRAPPFARLCCVSAALGMLWSPACLAFNLDVDKLTVYSGPEGSYFGYSLDFYIPDARTASVLVGAPKANTSQPDIVEGGAVYYCPWPSERSAQCKQIPFDTTNNRKIRVNGTKEPIEFKSNQWFGATVRAHKGKVVACAPLYHWRTLKPNPAKDPVGTCYVAIQNFSAYAEHSPCRNSNADPEGQGYCQAGFSLDFYKNGDLIVGGPGSFYWQGQVITVSIADIIANYSFKDILRKLAAEKQTDVAPASYDDSYLGYSVAAGEFTGDSQQELVAGIPRGAQNFGYVSIINSTDMTFIQNFTGEQMASYFGYTVVVSDVNNDGMDDILVGAPLFMEREFESNPREVGQVYLYLQASALLFQDPQVLTGTETFGRFGSSVAHLGDLNQDGYNDIAIGVPFAGKDQRGKVLIYNGNPRGLHSKPSQVLQGIWGSQTIPSGFGFSLRGDADIDKNDYPDLLVGAFGKGKVAVYRARPVVTVDAQLLLHPMIINLENKTCQIPEFPTPVACFSVRVCASIAGQSISNTIALLAEVQLDFLKQKGAIKRTLFLHNHQSHFTFPFVMKQQKSLHCQDFMVYLRDETEFRDKLSPINISLNYSLDDSTFKDSLEVKPILNHYRDNVVTEQAHILVDCGEDNLCVPDLKLSARPDKHQIIIGDENHLMLIINARNEGEGAYEAELFVIIPEEADYVGIERNNKGLRPLSCEYKMENVTRMVVCDLGNPMVTGTNFSLGLRFAVPRLEKTNMSINFDLQIRSSNKDNPDSNFERVQINITAIAQVEIRGVSHPPQIVLPIHNWEPEKKPHKEEEVGPLVEHIYELHNIGPSTISDSILDVGWPFSARDEFLLYIFHLQTLGPLQCQTNPEINPQDIKPAASPEDTPELSAFLRNATIPHLVRKRDVPVVQLHRQSPARILNCTNIDCLQISCAVGRLGGGESAVLKVRSRLWAHTFLKRKNDHYALASLVSFEVKKMPYKEQPAKLPAGSTAVKTSVIWATPNVSFSIPLWVIILAILLGLLVLAILTLALWKCGFFDRARPPQDEMTDREQLTSDKTPEA

SEQ ID NO: 3 - DNA, 6982 bp, Homo sapiens
acagacgtgcaaagccgtgaacgctgaggtcaggctgcagaggtgatgaagagataattgctttgcccctcctggtggtggggttggttttcacattctccatccaagctgcgcctttgacacaactggagcaagctctcgctccacctggggcattgcttctaaagcaataaatagcttgaggtgacagagacgaaactgattgtccttgagatgggactgcaatagaaatccgggcagcccgaagaggcacccagcgctccagccaccagctgggccgcccgggagtccctggctctagaccagccgcgaggaggcgccgcgagagagctggtccctgcccgcggccggaggagggctagagcccctgggccagccccccgagccggctgggcgggcgggcgggtgggagcagacgccgggcactgtcaccacgggtgcgccgagcgcaccgacccgggacacgggcagctggggaccgccagattccaccagccccccttgccccgcaggggtcctcggctcgcgctcctgggtagcagccacccaccggggcggagggagatgtcgcccggggccagccgcggtccccggggaagccaggcgccgctgatcgcgcccctctgctgcgccgcggccgcgctggggatgttgctgtggtcccccgcctgtcaggcgttcaacctggacgtggaaaagctcacagtgtacagcggccccaagggcagctacttcggctacgccgtggacttccacatacccgacgcccgcacagcgagtgtcttggtgggggcgcccaaagccaacaccagccagcccgatatcgtggaagggggagccgtctattactgtccttggcccgcggaggggtctgcgcagtgcaggcagataccgtttgacaccaccaacaacagaaagatcagagttaatggaaccaaagaacctatcgagttcaaatccaatcagtggtttggagcaacagtgaaagctcacaaaggaaaagttgtggcctgtgctcctttatatcactggagaactcttaaaccgacaccagaaaaggacccagttggcacctgctatgtagcaattcagaacttcagcgcctatgccgagttctctccttgccggaacagcaatgctgatccggaaggccagggttactgccaagcaggatttagtctggatttttataagaatggagaccttattgtgggaggacctgggagtttctactggcaaggacaagtgatcactgccagtgttgcagatatcattgcaaattactcattcaaggatatcctcaggaaactggcaggagaaaagcagacggaagtggctccagcttcctatgatgacagttaccttggatactcagttgctgctggggagtttactggggattctcagcaagaattggttgctggaattccaagaggagcacagaattttggatatgtttccatcattaactctacggatatgacgtttattcagaatttcacgggagaacagatggcatcttattttggatataccgttgtcgtatcagatgttaacagtgatggactggatgatgtcctggttggggcacctctctttatggaacgtgaatttgagagcaaccccagagaagtagggcaaatctacctgtatttgcaagtgagctctctcctcttcagagacccccagatcctcactggcaccgagacgtttgggagattcggtagtgctatggcacacttaggagacctgaaccaagatggatacaatgacattgccatcggagtgccttttgcaggcaaggatcaaagaggcaaagtgctcatttataatgggaacaaagatggcttaaacaccaagccttcccaagttctgcaaggagtgtgggcctcacatgctgtcccttccggatttggctttactttaagaggagattcagacatagacaagaatgattacccagatttgattgtgggtgcatttggaacaggaaaagtcgctgtttacagagcaagaccggttgtgactgtagatgcccagcttctgctgcacccaatgattatcaatcttgaaaataaaacttgccaggttccagactctatgacatctgctgcctgcttttctttaagagtatgtgcatctgtcacaggccagagcattgcaaacacaatagtcttgatggcagaggtgcaattagattccctgaaacagaaaggagctattaaacggacgctcttccttgataaccatcaggctcatcgcgtcttccctcttgtgataaaaaggcagaaatcccaccagtgccaggatttcatcgtttaccttcgagatgaaactgaattccgagataaattatctccaatcaacattagtttgaattacagtttggacgaatccacctttaaagaaggcctggaagtgaaaccaatattgaactactacagagaaaacattgttagtgaacaggctcacattctggtggactgtggagaagacaatctgtgtgttcctgacttgaagctgtcggctagaccagataagcatcaggtaatcattggagatgaaaatcaccttatgctcataataaatgcaagaaatgaaggggaaggagcatatgaagctgaactctttgtaatgataccagaagaggcagattatgttggaatcgaacgcaacaacaagggatttcgaccactgagctgtgagtacaagatggaaaatgtaaccaggatggtggtgtgtgaccttgggaaccctatggtgtctggaacaaattattccctgggcctccgatttgcagttccacgtcttgagaaaacaaacatgagcattaacttcgatctccaaatcagaagttccaacaaggacaatccagacagcaattttgtgagcctgcaaatcaacatcactgctgtagcgcaggtggaaataagaggagtgtcacaccctccgcagattgttctgcccattcataactgggaaccagaagaggagccccacaaagaggaggaggttggaccattggtggaacatatttatgagctgcacaatattggaccaagtaccatcagtgacaccatcctggaggtgggctggcctttctctgcccgggatgaatttcttctctatattttccatattcaaactctgggacctctgcagtgccaaccaaatcctaatatcaatccacaggatataaagcctgctgcctccccagaggacacccctgagctcagcgcctttttgcgaaactctactattcctcatcttgtcaggaagagggatgtacatgtggtcgaattccacagacagagccctgcaaaaatactgaattgtacaaatatcgagtgtttacaaatctcctgtgcagtgggacgactcgaaggaggagaaagcgcagtcctgaaagtcaggtcacgattatgggcccacaccttcctccagagaaaaaatgatccctatgctcttgcatccctggtgtcctttgaagttaagaagatgccttatacagatcagccagcaaaactcccagaaggaagcatagtaattaagacatcagttatttgggcaactccgaatgtttccttctcaatcccattatgggtaataatactagcaatacttcttggattgttggttctcgccattttaaccttagctttatggaagtgtggattctttgacagagccagacctcctcaggaggacatgaccgacagggaacagctgacaaatgacaagacccctgaggcatgacaagaaaaaaaaagaagaccaaagacctcaaacactggtcctgttcaaagaaaaagaaagaacatgagggttaaaaatcaaagctttctgatacctgacagtgacccaggaaatggaggggaccctggaaacatcacctcatctacaccgcactttggagaaattgtcctgggtgcccagtgagccctgttggaaaaggaaacaccaaagctggagaagccatcagcaattgtggaagattttcttttgctttaatcgttctgtacttggcagacactttgaaatgcgtatgggaacgtaagttgcagtccagatcaatccagcataagttgcagagatgaaatgtctgaatcactatagaatttacaaggtgaactaaggtgaaatgactgatttactatagaacttagaactaagaccacaccatccaacactactgtatccaatggaacatttgacacctccctatggaaaagaaacatttctaaggacattatggctcagggagacaagcaatatgttgttggtactgtgaaagtacttttgaaaggacaaaaataatcttgtgagtgagggtgagagtttatttatttattttccatgtctctaggttgagaatctgtcatctagagaatttggaaattaagatttggttagctagttttcaaaatcactcactcagtttcctcctaagaaccactgtgtgcagataactcatttagcagagcttcttcagaatttagacagtgctaaggaaaggaagattggcattaggaatcagttctatttaataccttgctgccttccatctgaaattcctttcactcacattatccagggactcacatctgcttaatttagctttgcaagatgttgcatttatatttccttgtatatctttccattgacttagttaatacatgaatttcgaggtactctctacctttggagtgtttaaaagtcatggtaaaatgtctagagttcattaattaaaaagcaatttaacattatgggtaaacattatttttaatatctataagaagcaacacttcttatagatatttagcttgggattaaatacccgtttttcctgctatgccttgtacttgtatacacagagaaaacatgttgaatataacttagacaaaaacataaaattatacaggcatgagccaaactgggaaaactgattcctaaagatttttatttaaaattgcttattccaactttattacttatttttactgctaatctttataatatgtaactttggagaataatcttttatttaagctattttcccatcatcttgaatttaaccatcacaaattttttatgtttaaaactcttgtaaataattcataatcttgtaatattatcatttttgaagtaaaattgaccagccaaatttataggtagtctgcacaattttgtatccttttttaataatgaaaaattactatgaagaaatactgaacaaatttttatgtgcaatattttatagacctatgtatctgaagcatgtttacactggcgtttttttttttaattaatttcctaaatgttaagtatgatagaacaagctgacccaaatccttaagtttacaaagctgttggaaaactttgtgtcctgatttcaacaatcacgctttgtttgaaagatgagccaagctcacagacactaaattttatgtcatgccataagctggagaggagccatttggctacagctgcggaacttcattgaggagcaaatgaaaggcacatggacgagcacgctggtgcagttcatgttcttcctgcctgtgaattgaatactgtcctggtagcagtttcgggtcggtcaggagctcaaggctggtttgtgtggctgactacggatgagcactgaagttgcctcaaagaattaaggggtgtccacaccagcctctgggggtctttggtgttagtcttccaggtagagctggttttacaagtaggtggccatctacaggatgtgatgtgagcgatgccagacagctctctctgaccccaggtaatgccctgaatctggtgatcctggctgatctgtgaccaatagagattagctccttgggatttggggtcctaaaaggtccctgaaaaaatgcaccccttgtctttaagccaacattggtgaaggaactgagaactcttagggttacataaaaaaagacccctgttgagatagtttatgcagatacctggaaggaactagagatgccaggaggaagactgagacccagaatgaagcaaggcatgagggctccaaagtgaactgcaccatcccagagtctcagccaagatggccctttcctagagggacgggcaccttccacctgcccacaggcactgctacctatgggaattgcagaaggccgttgtacacacgcgcatgcacgcagtggccctctgccctttagaatatggaagcaaagtctgggtgcagtgactcacacttgtaatcccaacactttgggaggccgagacgggcagatcacctgagttcaggagttcaagaccagcctggccaacgtggcaaaaccccatctctactaaaaatacaaaaattagctgggcgtggtggcagtctcctgtaatcccagctacctaggaggctgaggcagagagagaatcgcttgaacccaggaggcggaggttgcagtgagctgagatcgcgcgactgcactccagcctgggcgacagagggaaactctgtctcaacaaaaaaataaataaaataaaaaataaaaaaagaatatggaagcaaatggagaaagggaggaaactgaggtcaggcaaaccttccagcaatccgttctgcccagagaacatactgacgacagagaacacactgagcgttatctgcttctagttagagcattgccgattgccttcataattcataatgtgctgtgctcctctgagggtctatctgtatttctcttcaaagaattccctttttaaattcttgcagctgggttttaagacacaataatggggcaaattaagttttataaacttggagatttttatacagtaaataaaaaaataatatgtactagtcacccctgaaactgcattcttagaactcgactcttgttttatttaaatacgtgcgtagatatctgtgaagattttcatgtacctatttaccttgtcactaataaaattaaaactgaaagacttgaaaaaaaaaaaaaaaaaa

SEQ ID NO: 4 - DNA, 5782 bp, Mus musculus
gcacagggcgagaggactgtcactgtggacgctcctagctcaccgacccgggacaccggcctctggggagcgtcaggccttctcctctgggctccacggctcccgatcccgggtgaacagcgttcgggaaggagggcgatgtctgcgggaacccactgtggtcccccggggaaccgggcacctccgttcgcgcgtctctgttgcgtctcggccgcgctggggatgctgtggtcccctgcgtgtctggcgttcaacttggatgtggacaagctcactgtgtacagtggccccgagggcagctacttcggctactcactggacttctacatacctgatgcgcgcacagccagtgtcttagtgggggcacccaaagccaacactagccagccggatatcgtagaagggggagctgtctactactgtccctggccctcagagaggtctgcacagtgcaagcagataccgtttgacaccaccaacaacaggaaaatcagagttaatggaaccaaagaacctatagaatttaaatcaaaccagtggtttggagccacagtgagagctcacaaaggaaaagttgtggcttgtgcccctttgtatcactggagaactctgaaacctaatccagcgaaggacccagttggcacatgctatgtagcaattcagaatttcagtgcctatgctgagcactcaccttgtcgaaacagcaatgctgatcccgaaggccaaggttactgccaggcagggtttagcctagacttctataagaatggagaccttatagtgggaggacctggaagtttttactggcaaggtcaggtgatcactgtcagtatagcagacatcattgcaaattactcattcaaggatattctacgaaaattggccgcagaaaagcagacagacgtggctccagcttcctatgatgacagctaccttggatattcggtcgctgctggagaattcactggggactctcagcaagaattggtggctgggattccaagaggagcacagaactttggatatgtctccatcattaactccacagacatgaccttcattcagaattttactggtgagcagatggcatcttatttcggatatactgttgtggtatcagatgttaacaatgatggcatggatgacatattggtcggggcacctctctttatggagcgagaatttgaaagtaaccctagagaagtgggacaggtctacttgtaccttcaagcaagcgccctcctcttccaagacccacaggtcctcaccggcacggagacatttgggagatttggtagctctgtggcccacttgggggacctgaaccaagatggatacaatgacatcgccatcggtgtgccctttgcaggcaaggatcaacgaggtaaagtcctcatttacaatggaaacccaagaggtttacacagcaagccttcccaagttctgcaaggaatatgggggtcacagaccatcccttctggatttggcttttctctaagaggagatgcagacatagacaagaatgattacccagatttgcttgtaggggcatttggaaaagggaaagtagctgtttacagagcaaggccagttgtaaccgtggatgcccagcttctgctgcacccgatgattatcaaccttgaaaataaaacttgccagattccagaattccctactcctgtggcctgcttttctgtaagagtctgtgcatctatagcaggccagagcatttcaaatacaatagccctgctggccgaggtgcagttagatttcctgaagcaaaaaggagccatcaaacggacgctctttctccacaaccaccagtcccatttcaccttcccctttgtgatgaagcagcagaaatccctccactgccaggattttatggtttaccttcgggatgaaactgaattccgagataaattgtctccaatcaacatcagcctgaactacagtttggatgattctacctttaaagacagcctggaagtgaagccaattttgaaccactacagggacaatgtagttactgagcaggctcacatcctggtggactgtggagaagacaatctatgcgttcctgacttgaagctgtcagctagaccagataagcatcagataattattggcgatgaaaatcacctaatgctcataataaatgcaagaaatgagggagaaggggcctacgaagccgaactctttgttatcatccccgaagaagcagattatgttgggattgagcgcaacaacaagggactgaggcccctgagttgtgagtacaagatggaaaacgtgaccaggatggtggtgtgtgaccttgggaacccgatggtgactggaacaaatttttctctgggcctccgatttgctgttcctcgccttgagaaaacaaatatgagcattaacttcgatctccaaatcagaagctccaataaggacaatcctgacagcaacttcgagcgtgtacagatcaacatcactgccattgctcaggtcgaaatcagaggagtgtcccaccctccgcagattgttctgcccatccacaactgggaaccagaaaagaagccacacaaagaggaggaagtcggtcctttggttgagcatatttatgagctgcacaatattggacccagcaccatcagtgacagcattcttgacgtgggctggccgttctctgcacgggatgagtttctcctctatatttttcatcttcaaactctgggacctctacaatgtcaaacaaatcctgagatcaacccacaggatataaagcctgctgcctccccagaggatacccctgagctcagtgccttcttaagaaatgctacaattccccatcttgtcagaaagagggatgtgccagtggtccagctccacagacagagccctgcaagaatactgaactgtaccaacattgactgcttgcaaatctcttgtgcagtgggtcgcctgggaggaggcgaaagtgcagtcctaaaggtcagatcgagattgtgggcccacacgttcctcaagagaaagaatgatcattatgctcttgcatccctggtgtcctttgaagtcaagaagatgccgtataaagaacagccagcaaagctgccagcggggagcacagcggttaagacgtcggttatttgggccactccgaatgtctccttctcaattccattgtgggtcataatactagcgatccttcttggtttgctggttctggctatcttaactttagctttatggaagtgtggattctttgatagagcaagacctcctcaggatgaaatgacagacagggaacaactgaccagtgacaagaccccagaggcgtgacttggaaacatgaagacctaaaattcaagcactggtcctattcaaagaaagaaagggaggaaggacacaagggctcaatgatcatagccttccatacctgccaatgacccagggaatgaagagtccctaatgatcacctcatctaagtcatgcattggtgtggagtgcagagaaccccacgtgaaaagaaatgcagccactaacactacagaaacaatccacagccatgaagatttcctttgttcttaccgtttcacatctggctgacattctggaacagctgtaggagcttaggcagctgtctaggtcagcccaatatgacttacaaaaacaattcctacagagatgagatgaactgagattgaacactactgtaatccgatggaatctcagacacctccctatggaatagaaccctttctaaggatgcgaaggctcagggagaaaagtgatatgatgttgggactatgaaagtgcttatggaactacagagatgatttcatcatgggaagaccaaaaatatatttatttatgtttcctttctctaggtagagaattgggccatttagaggatgtggaaattaagatttggttagctagatttcatcactactcagtttacttcctcttaagataactactctgtgcagatagcatatttaattgggcttcttcaggtgttagatggtgttacacatggtttggcattagggctctgttgtgcttaattttctgctgcctcgagtgacttttcttcatgttctctggaaactcatgtctgcataattcaactttacaaaatcttatatttatagtttcttttatatgtgtaaaaaactatttccagtgacttctgtaaagcattcatttcaaggtactcactatgctgatgatgttgaaaagtacttgcacaattttgtagagtccatataaaaagcaatttaactatatgggtgaatattatcttaatagctatagtaagcaccaatatttaccttgaaatttacacactcattttcattgtaatacctcacatttatataaaagagaaaactgtgttgaacataaataagatgaagatatataaagttatacaagtgtgagccaaacagaaactcactcttaaggacatagcagtgcttattacagctttattgtccactctttatggttaacttttcctagcaagtaatcttggagaatattttatatttaagctattttaccatcatctttacctaaacattgcttagagctcttgtaaataatccataataatttgtaatgttgtcattttgaagtaatattgaccagccaaattaataagattgtctgcacaattttatatcctttttataataatgaaaaattgctatggagaaatgctgaacaaaatgttatgtgcaatattttatagacctatgtatctgaggcatgtttacactggcattagtttcttttttttaattaatatcctgatcactaagtctgatataacaagctgaccaaaatcctaacgtttacaaaacagaaaactctgtctcagtttcaacaatgacactttatttgaaagatgacccaaactcacagacagtaaattttatgtggtgccttaacctggaaaggagccatttggcttcagttgctacagttccttggaaagcaaatgaaatgcacagagtcaaacagcattgctgttcatcttgccctgcttattagttaaatctgtcctggtagctggcttatgtctataagggctagtgtctaggagtacgacacttgcctcaaagagtcacacaccagcctctggggttgtctttactgttagtctttttggtgggacaactcactctacattttaatttttatgattcctgatgtggatccattacaaacttctcagaagtttggagtgcatctctgatcttgaaagaactcagagaactcttagggtgaaatgggaactgtgggagacaacccatatggggaaaactaaagatgtcaggggggcgacaagaccaacacagaagaaggtatgggaatatcagaggcaaaacatggtttcccatggtctcaactgtagaagtgcaggagggctatcatacacatgcacacagaagctgaggtcaggtgacccctctggggacatgttcataggcatcccatcctcacagagaactgagtattcaacaatgctcatggcattgctgatgcttctgtcatacattggtattctcgaggagtctatctgtatttctctttaatgagttctttcctagttctcacagctgtttttataagacagagggaaatgagcagattttctaatcttggagatttttatacagtaaagcattctagttgtcactgaaatctaaatcttagaattcttgtcttatttaaatacatgtatagagacagtgtagattttcatgtacctatttacattgtcacc

SEQ ID NO: 5 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK